(19) United States
(12) Patent Application Publication
(10) Pub. No.: US 2016/0183659 A1
(43) Pub. Date: Jun. 30, 2016

(54) RESIN COMPOSITION FOR LASER ENGRAVING, FLEXOGRAPHIC PRINTING ORIGINAL PLATE FOR LASER ENGRAVING, METHOD FOR PRODUCING FLEXOGRAPHIC PRINTING ORIGINAL PLATE FOR LASER ENGRAVING, FLEXOGRAPHIC PRINTING PLATE, AND METHOD FOR MAKING FLEXOGRAPHIC PRINTING PLATE

(71) Applicant: FUJIFILM Corporation, Tokyo (JP)

(72) Inventor: Hiroshi TASHIRO, Haibara-gun (JP)

(73) Assignee: FUJIFILM Corporation, Tokyo (JP)

(21) Appl. No.: 15/064,722

(22) Filed: Mar. 9, 2016

Related U.S. Application Data
(63) Continuation of application No. PCT/JP2014/075368, filed on Sep. 25, 2014.

(30) Foreign Application Priority Data
Sep. 26, 2013 (JP) 2013-199606

(51) Int. Cl. A45D 31/00; A61K 8/19; A61K 8/91; A61Q 3/02; A61Q 3/04

(52) U.S. Cl. CPC A45D 31/00; A61Q 3/02; A61Q 3/04; A61K 8/91; A61K 8/19; A61K 2800/884; A61K 2800/81

(57) ABSTRACT

Provided are a resin composition for laser engraving, from which a flexographic printing plate having excellent rinsability of the engraving residue generated by laser engraving, as well as excellent engraving sensitivity, printing durability and printing durability over time can be obtained; a flexographic printing plate precursor for laser engraving produced using the resin composition for laser engraving; a method for making a flexographic printing plate using the flexographic printing plate precursor; and a flexographic printing plate obtained by the plate-making method.

Disclosed is a resin composition for laser engraving of the present invention, including (Component A) a resin that is a plastomer, (Component B) a vulcanizing agent, and (Component C) a compound having a hydrolyzable silyl group and/or a silanol group in an amount of 0 parts by mass or more and less than 0.1 parts by mass relative to 100 parts by mass of Component A.

### CROSS-REFERENCE TO RELATED APPLICATIONS

This application is a Continuation of PCT International Application No. PCT/JP2014/075368 filed on Sep. 25, 2014, which claims priority under 35 U.S.C. §119(a) to Japanese Patent Application No. 2013-199606 filed on Sep. 26, 2013. The contents of these applications are incorporated herein by reference in their entirety.

### BACKGROUND OF THE INVENTION

1. Field of the Invention

The present invention relates to a resin composition for laser engraving, a flexographic printing plate precursor for laser engraving, a method for producing a flexographic printing plate precursor for laser engraving, a flexographic printing plate, and a method for making a flexographic printing plate.

2. Description of the Related Art

There have been many proposals relating to the so-called “direct engraving CTP method”, in which plate-making is achieved by directly engraving a relief forming layer by means of a laser. In this method, concave sections are formed by directly irradiating a flexographic printing plate precursor with laser light, and causing thermal decomposition and volatilization to occur as a result of photothermal conversion. Unlike relief formation using an original image film, the direct engraving CTP method enables the relief shape to be freely controlled. For this reason, in a case in which an image such as an outlined character is formed, the image region can be engraved more deeply than other regions, or in the case of a fine halftone dot image, shouldered engraving or the like can also be carried out in consideration of the resistance to printing pressure. Furthermore, regarding the laser used for this method, a high power carbon dioxide laser is frequently used. In the case of a carbon dioxide laser, all organic compounds can absorb the irradiation energy and convert the energy to heat. On the other hand, inexpensive small-sized semiconductor lasers have been developed; however, since these emit visible and near-infrared light, organic compounds which absorb the laser light and convert the light to heat are needed.

Regarding the conventional flexographic printing plate precursors for laser engraving, for example, those described in JP2008-229875A and JP2009-23181 A are known. JP2008-229875A describes a flexographic printing plate precursor which has excellent elasticity and from which the engraving residue generated after laser engraving can be easily removed by washing with water, in which the flexographic printing plate precursor is formed by laminating (A) a support layer, (B) a resin layer formed from an elastomeric composition including an elastomeric binder and a crosslinking agent capable of crosslinking this elastomeric binder, and (C) a hydrophilic resin layer.

JP2009-23181A describes a flexographic printing plate precursor which undergoes less shape deterioration caused by laser engraving, the printing plate precursor including a flexible support, and a resin composition layer formed on the support, in which the resin composition layer can be laser-engraved, and has a Shore D hardness in the range of 45° to 95°.

### SUMMARY OF THE INVENTION

An object of the present invention is to provide a resin composition for laser engraving, from which a flexographic printing plate having excellent rinsability of the engraving residue generated by laser engraving, as well as excellent engraving sensitivity, printing durability and printing durability over time can be obtained; a flexographic printing plate precursor for laser engraving produced using the resin composition for laser engraving; a method for making a flexographic printing plate using the flexographic printing plate precursor; and a flexographic printing plate obtained by the plate-making method.

The object of the invention has been addressed by the following means <1>, <11>, <12>, <14>, <17>, and <19>. These are listed together with preferred embodiments <2> to <10>, <13>, <15>, <16>, and <18>.

<1> A resin composition for laser engraving including (Component A) a resin that is a plastomer; (Component B) a vulcanizing agent; and (Component C) a compound having a hydrolyzable silyl group and/or a silanol group in an amount of 0 parts by mass or more and less than 0.1 parts by mass relative to 100 parts by mass of Component A.

<2> The resin composition for laser engraving according to <1>, in which Component A is a poly(conjugated diene)-based synthetic resin.

<3> The resin composition for laser engraving according to <1> or <2>, in which the content of Component B in the resin composition is 10 parts by mass to 20 parts by mass relative to 100 parts by mass of Component A.

<4> The resin composition for laser engraving according to any one of <1> to <3>, in which Component A is one or more resins selected from the group consisting of polybutadiene, polyisoprene, and polyisobutylene.

<5> The resin composition for laser engraving according to any one of <1> to <4>, further including (Component D) a polymerizable monomer, and (Component E) a polymerization initiator.

<6> The resin composition for laser engraving according to <5>, in which Component E is a thermal polymerization initiator.

<7> The resin composition for laser engraving according to <5> or <6>, in which Component E is an organic peroxide.

<8> The resin composition for laser engraving according to any one of <1> to <7>, further including (Component F) a vulcanization accelerating agent.

<9> The resin composition for laser engraving according to any one of <1> to <8>, further including (Component G) a photothermal conversion agent.

<10> The resin composition for laser engraving according to <9>, in which Component G is carbon black.

<11> A flexographic printing plate precursor for laser engraving, having a relief forming layer formed from the resin composition for laser engraving according to any one of <1> to <10>, on a support.

<12> A flexographic printing plate precursor for laser engraving, having a crosslinked relief forming layer obtained by crosslinking a relief forming layer formed from the resin composition for laser engraving according to any one of <1> to <10> by means of heat and/or light, on a support.

<13> The flexographic printing plate precursor for laser engraving according to <12>, including a crosslinked relief forming layer crosslinked by heat.

<14> A method for producing a flexographic printing plate precursor for laser engraving, the method including a layer forming step of forming a relief forming layer formed from the resin composition for laser engraving according to any one of <1> to <10>; and a crosslinking step of crosslinking the relief forming layer by means of heat and/or light, and thereby obtaining a flexographic printing plate precursor having a crosslinked relief forming layer.

<15> The method for producing a flexographic printing plate precursor for laser engraving according to <14>, in which in the crosslinking step, the relief forming layer is crosslinked by means of heat.

<16> The method for producing a flexographic printing plate precursor for laser engraving according to <14> or <15>, in which in the crosslinking step, crosslinking is performed in a state in which at least the central section of the surface of the relief forming layer is covered with a material capable of blocking air.

<17> A method for making a flexographic printing plate, the method including: a step of preparing the flexographic printing plate precursor for laser engraving according to any one of <11> to <13>; and an engraving step of laser-engraving the flexographic printing plate precursor for laser engraving, and thereby forming a relief layer.

<18> The method for making a flexographic printing plate according to <17>, further including, after the engraving step, a rinsing step of rinsing the surface of the relief layer using an aqueous rinsing liquid, after the engraving step.

<19> A flexographic printing plate produced by the plate-making method according to <17> or <18>.

According to the present invention, a resin composition for laser engraving, which gives a flexographic printing plate having excellent rinsability of the engraving residue generated by laser engraving, as well as excellent engraving sensitivity, printing durability and printing durability over time, a flexographic printing plate precursor for laser engraving, a method for producing a flexographic printing plate precursor for laser engraving, a flexographic printing plate, and a method for making a flexographic printing plate, can be provided.

### DESCRIPTION OF THE PREFERRED EMBODIMENTS

The resin composition for laser engraving of the invention (hereinafter, also simply referred to as “resin composition”) includes (Component A) a resin that is a plastomer; (Component B) a vulcanizing agent; and (Component C) a compound having a hydrolyzable silyl group and/or a silanol group, in an amount of 0 parts by mass or more and less than 0.1 parts by mass relative to 100 parts by mass of Component A.

Meanwhile, according to the invention, the description of “lower limit to upper limit”, which indicates a value range, means “the lower limit or higher, and the upper limit or lower”, and the description of “upper limit to lower limit” means “the upper limit or lower, and the lower limit or higher”. That is, the description indicates a value range including the upper limit and the lower limit. Also, according to the invention, “(Component A) a resin that is a plastomer” or the like is also simply described as “Component A” or the like.

A combination of preferred embodiments in the following description constitutes a more preferred embodiment.

Hereinafter, the essential or optional constituent components of the resin composition of the invention will be explained.

(Component A) Resin that is Plastomer

The resin composition of the invention includes (Component A) a resin that is a plastomer, as an essential component.

The term “plastomer” according to the invention means, as described in “Shinpan Kobunshi Jiten (Polymer Dictionary, New Edition)” compiled by the Society of Polymer Science, Japan, (Asakura Publishing Co. Ltd., Japan, published in 1988), a macromolecular body which has a property of easily undergoing fluid deformation when heated and being capable of solidifying into a deformed shape when cooled. The term plastomer is a term opposed to the term elastomer (a substance having a property by which, when an external force is applied thereto, the substance is instantaneously deformed in response to the external force, and when the external force is removed, the substance restores the original shape in a short period of time), and a plastomer does not exhibit such elastic deformation as that exhibited by an elastomer, and easily undergoes plastic deformation.

According to the invention, a plastomer means a substance which, when the original size is designated as 100%, can be deformed up to 200% of the original size by a small external force at room temperature (20° C.), and even if the external force is removed, does not have its size restored to 130% or less of the original size. A small external force specifically refers to an external force with a tensile strength of 1 MPa to 100 MPa. More particularly, a plastomer means a polymer with which, based on the tensile permanent strain test of JIS K 6262-1997, a dumbbell-shaped No. 4 specimen as defined by JIS K 6251-1993 can be extended to twice the gauge length before pulling, without breaking, in a tensile test at 20° C., and the tensile permanent strain measured after maintaining the specimen for 60 minutes in a state of being extended to twice the gauge length before pulling, removing the external tensile force, and maintaining the specimen for 5 minutes, is 30% or more. Incidentally, in this invention, the entire procedure was performed according to the tensile permanent strain test method of JIS K 6262-1997, except that the specimen was made into a dumbbell shape as defined by JIS K 6251, the time for maintaining the specimen was set to 60 minutes, and the temperature of the test chamber was adjusted to 20° C.

Meanwhile, in the case of a polymer which cannot be subjected to the measurement described above, that is, a polymer which is deformed even if an external tensile force is not applied during a tensile test and does not restore its original shape, or a polymer which breaks when a small external force is applied at the time of the measurement described above, the polymer corresponds to a plastomer.

Furthermore, the resin that is a plastomer of the present invention is such that the glass transition temperature (Tg) of the polymer is lower than 20° C. In the case of a polymer having two or more Tg's, all of the Tg's are lower than 20° C. Incidentally, the Tg of a polymer can be measured by differential scanning calorimetry (DSC).

Examples of Component A as such include a polyolefin resin such as polyethylene or polyisobutylene, and a poly(conjugated diene)-based resin such as polybutadiene, hydrogenated polybutadiene, polyisoprene, or hydrogenated polyisoprene. Among them, a poly(conjugated diene)-based resin is preferred; a poly(conjugated diene)-based resin having a so-called internal olefin bond is more preferred; polybutadiene or polyisoprene is even more preferred; and a poly(conjugated diene)-based synthetic resin is preferred. Polybutadiene or polyisoprene may be partially or completely hydrogenated.

The polybutadiene described above may be any polymer having butadiene as a monomer unit in the main chain, and examples thereof include a terminal-modified polybutadiene, a partially hydrogenated polybutadiene, and a hydrogenated polybutadiene. Commercially available polybutadiene, polybutadiene polyol, and the like may also be used, and examples include KURAPRENE LBR series (manufactured by Kuraray Co., Ltd.), POLY BD (manufactured by Idemitsu Kosan Co., Ltd.), UBEPOL series (manufactured by Ube Industries, Ltd.), and NIPOL (registered trademark) BR series (manufactured by Zeon Corporation.).

Furthermore, it is known that butadiene is polymerized by 1,2-addition or 1,4-addition depending on the catalyst or the reaction conditions; however, a polybutadiene polymerized by any of the aforementioned additions may be used for the present invention. Among these, it is more preferable that 1,4-polybutadiene is a main component.

Meanwhile, the content of 1,4-polybutadiene is preferably 50% by mass or more, more preferably 65% by mass or more, even more preferably 80% by mass or more, and particularly preferably 90% by mass or more.

Also, the contents of the cis-form and the trans-form are not particularly limited; however, from the viewpoint of exhibiting rubber elasticity, it is preferable that the content of cis-1,4-polybutadiene is 50% by mass to 98% by mass, more preferably 65% by mass to 98% by mass, even more preferably 80% by mass to 98% by mass, and particularly preferably 90% by mass to 98% by mass.

The polyisoprene described above may be any polymer having isoprene as a monomer unit in the main chain, and examples thereof include a terminal-modified polyisoprene, a partially hydrogenated polyisoprene, and a hydrogenated polyisoprene. Commercially available polyisoprene, polyisoprene polyol, and the like may also be used, and examples include KURAPRENE LIR series (manufactured by Kuraray Co., Ltd.) and NIPOL (registered trademark) IR series (Zeon Corporation.).

Furthermore, it is known that isoprene is polymerized by 1,2-addition or 1,4-addition depending on the catalyst or the reaction conditions; however, a polyisoprene polymerized by any of the aforementioned additions may be used for the present invention. Among these, from the viewpoint of obtaining a desired Mooney viscosity, it is more preferable that 1,4-polyisoprene is a main component.

Meanwhile, the content of 1,4-polyisoprene is preferably 50% by mass or more, more preferably 65% by mass or more, even more preferably 80% by mass or more, and particularly preferably 90% by mass or more.

Furthermore, the contents of the cis-form and the trans-form are not particularly limited; however, from the viewpoint of exhibiting rubber elasticity, it is preferable that the content of cis-1,4-polyisoprene is 20% by mass to 98% by mass, more preferably 30% by mass to 80% by mass, even more preferably 30% by mass to 60% by mass, and particularly preferably 30% by mass to 40% by mass.

The polyisobutylene may be any polymer having isobutylene as a monomer unit in the main chain, and examples thereof include terminal-modified polyisobutylene. Commercially available polyisobutylene, polyisobutylene polyol, and the like may also be used, and examples include EPION series (manufactured by Kaneka Corporation.).

Furthermore, the polybutadiene, the polyisoprene, and the polyisobutylene as described above are all such that the number average molecular weight is preferably 1,500 to 500,000, more preferably 2,000 to 300,000, and even more preferably 2,500 to 250,000.

Meanwhile, the number average molecular weight (Mn) and the weight average molecular weight (Mw) of a resin or the like according to the invention can be determined by, for example, measuring the molecular weights using gel permeation chromatography (GPC), and calculating the corresponding values by calibrating the measured values relative to polystyrene with known molecular weights.

In the resin composition for laser engraving of the invention, only one kind of Component A may be used, or two or more kinds thereof may be used in combination.

The content of Component A is preferably 10% by mass to 95% by mass, more preferably 15% by mass to 70% by mass, and even more preferably 20% by mass to 65% by mass, relative to the total solid content mass of the resin composition. When the content of Component A is in the above-described range, it is preferable because a flexible relief layer having excellent rinsability of the engraving residue is obtained.

Furthermore, as Component A, a commercially available product can also be used, and examples thereof include UBEPOL BRI50L (manufactured by Ube Industries, Ltd.), and LBR-305 and LIR-30 (all manufactured by Kuraray Co., Ltd.).

(Component B) Vulcanizing Agent

Furthermore, the resin composition of the invention includes (Component B) a vulcanizing agent as an essential component.

In regard to the vulcanization of a rubber composition, it is known that a vulcanization reaction can be efficiently induced by performing vulcanization (crosslinking) using a vulcanizing agent and a vulcanization accelerating agent in combination. Regarding the vulcanizing agent, elemental sulfur (free sulfur) or a sulfur-donating organic vulcanizing agent is used.

Regarding the sulfur-donating organic vulcanizing agent, morpholine sulfide, dithiodicaprolactam, alkylphenol disulfide, a polymeric polysulfide, and the like are used.

Component B according to the invention is not particularly limited, and examples thereof include elemental sulfur and an organic vulcanizing agent. However, elemental sulfur is preferred.

Furthermore, the content of Component B is preferably 5 parts by mass to 30 parts by mass, and more preferably 10 parts by mass to 20 parts by mass, relative to 100 parts by mass of Component A in the resin composition.

Regarding Component B, commercially available products can also be used, and examples thereof include sulfur (manufactured by Tsurumi Chemical Industry Co., Ltd.) and VULNOC R (manufactured by Ouchi Shinko Chemical Industrial Co., Ltd., morpholine disulfide).

(Component C) Compound Having Hydrolyzable Silyl Group and/or a Silanol Group

The resin composition of the invention includes (Component C) a compound having a hydrolyzable silyl group and/or a silanol group, in an amount of 0 parts by mass or more and less than 0.1 parts by mass relative to 100 parts by mass of Component A.

It is preferable that the resin composition of the invention includes Component C in an amount of less than 0.1 parts by mass relative to 100 parts by mass of Component A, or does not include Component C, and it is more preferable that the resin composition includes Component C in an amount of less than 0.1 parts by mass.

The “hydrolyzable silyl group” for Component C is a silyl group having a hydrolyzable group, and examples of the hydrolyzable group include an alkoxy group, an aryloxy group, a mercapto group, a halogeno group, an amide group, an acetoxy group, an amino group, and an isopropenoxy group. The silyl group is hydrolyzed to become a silanol group, and the silanol group is dehydrated and condensed to produce a siloxane bond. Such a hydrolyzable silyl group or silanol group is preferably represented by the following Formula (1).

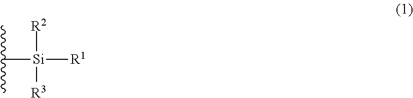

In the above Formula (1), at least any one of R¹ to R³ represents a hydrolyzable group selected from the group consisting of an alkoxy group, a mercapto group, a halogen atom, an amide group, an acetoxy group, an amino group and an isopropenoxy group, or a hydroxyl group. The others of R¹ to R³ each independently represent a hydrogen atom, a halogen atom, or a monovalent organic substituent (examples thereof include an alkyl group, an aryl group, an alkenyl group, an alkynyl group, and an aralkyl group). Also, the dotted line part represents a position of bonding to another structure.

In a case in which R¹ to R³ each represent a monovalent organic group, a preferred organic group is an alkyl group having 1 to 30 carbon atoms from the viewpoint that solubility in various organic solvents can be imparted.

In the above Formula (1), a hydrolyzable group that is bonded to a silicon atom is particularly preferably an alkoxy group or a halogen atom.

The alkoxy group is preferably an alkoxy group having 1 to 30 carbon atoms, more preferably an alkoxy group having 1 to 15 carbon atoms, even more preferably an alkoxy group having 1 to 5 carbon atoms, and particularly preferably an alkoxy group having 1 to 3 carbon atoms.

Furthermore, examples of the halogen atom include a F atom, a Cl atom a Br atom, and an I atom, and from the viewpoints of the ease of synthesis and stability, a Cl atom and a Br atom are preferred, while a Cl atom is more preferred.

Component C according to the invention is preferably a compound having at least one or more groups each represented by Formula (1) described above, and more preferably a compound having at least two or more groups each represented by Formula (1) described above. Also, as Component C, a compound having at least two hydrolyzable silyl groups is particularly preferably used.

Furthermore, as Component C, a compound having two or more silicon atoms in the molecule is preferably used. The number of silicon atoms contained in the compound is preferably from 2 to 6, and most preferably 2 or 3.

Regarding the hydrolyzable group, the number of hydrolyzable groups that can be bonded to one silicon atom is in the range of 1 to 3, and the total number of hydrolyzable groups in Formula (1) is preferably in the range of 2 or 3, and it is particularly preferable that three hydrolyzable groups are bonded to a silicon atom. When two or more hydrolyzable groups are bonded to a silicon atom, they may be identical to or different from each other.

Specific examples of the alkoxy group include a methoxy group, an ethoxy group, a propoxy group, an isopropoxy group, a butoxy group, a tert-butoxy group, a phenoxy group, and a benzyloxy group. These various alkoxy groups may be used in combination of plural groups, or a plural number of different alkoxy groups may be used in combination.

Examples of an alkoxysilyl group having an alkoxy group bonded thereto include trialkoxysilyl groups such as a trimethoxysilyl group, a triethoxysilyl group, a triisopropoxysilyl group, and a triphenoxysilyl group; dialkoxymonoalkylsilyl groups such as a dimethoxymethylsilyl group and a diethoxymethylsilyl group; and monoalkoxydialkylsilyl groups such as a methoxydimethylsilyl group and an ethoxydimethylsilyl group.

Specific examples of the aryloxy group include a phenoxy group. Examples of an aryloxysilyl group having an aryloxy group bonded thereto include triaryloxysilyl groups such as a triphenoxysilyl group.

It is preferable that Component C has at least a sulfur atom, an ester bond, a urethane bond, an ether bond, a urea bond, or an imino group.

Among them, it is preferable from the viewpoint of crosslinkability that Component C contains a sulfur atom, and from the viewpoint of removability (rinsability) of the engraving residue, it is preferable that Component C contains an ester bond, a urethane bond, or an ether bond (particularly, an ether bond contained in an oxyalkylene group), which are easily decomposed by alkaline water. Component C containing a sulfur atom functions as a vulcanizing agent or a vulcanization accelerating agent at the time of a vulcanization treatment, and accelerates a reaction (crosslinking) of the (B) polymer containing a conjugated diene monomer unit. As a result, the rubber elasticity required by a printing plate is exhibited. Also, the strength of the crosslinked relief forming layer and the relief layer is increased.

Furthermore, it is preferable that Component C according to the invention is a compound which does not have an ethylenically unsaturated bond.

Component C according to the invention may be a compound in which plural groups each represented by Formula (1) described above are bonded via a divalent linking group, and such a divalent linking group is preferably a sulfide group (—S—), an imino group (—N(R)—), or a linking group having a urethane bond (—OCON(R)— or —N(R)—COO—) from the viewpoint of effectiveness. Meanwhile. R represents a hydrogen atom or a substituent. Examples of the substituent for R include an alkyl group, an aryl group, an alkenyl group, an alkynyl group, and an aralkyl group.

The synthesis method for Component C is not particularly limited, and Component C can be synthesized by a known method. A representative synthesis method for Component C containing a linking group having the particular structure is described below as an example.

<Synthesis Method for Compound Having Sulfide Group as Linking Group and Having Hydrolyzable Silyl Group and/or Silanol Group>

The synthesis method for Component C having a sulfide group as a linking group (hereinafter, appropriately referred to as “sulfide linking group-containing Component C”) is not particularly limited. However, specific examples thereof include synthesis methods such as a reaction between Component C having a halogenated hydrocarbon group and an alkali sulfide; a reaction between Component C having a mercapto group and a halogenated hydrocarbon; a reaction between Component C having a mercapto group and Component C having a halogenated hydrocarbon group; a reaction between Component C having a halogenated hydrocarbon group and a mercaptan; a reaction between Component having an ethylenically unsaturated double bond and a mercaptan; a reaction between Component C having an ethylenically unsaturated double bond and Component C having a mercapto group; a reaction between a compound having an ethylenically unsaturated double bond and Component C having a mercapto group; a reaction between a ketone and Component C having a mercapto group; a reaction between a diazonium salt and Component C having a mercapto group; a reaction between Component C having a mercapto group and an oxirane; a reaction between Component C having a mercapto group and Component C having an oxirane group; a reaction between a mercaptan and Component C having an oxirane group; and a reaction between Component C having a mercapto group and an aziridine.

<Synthesis Method for Compound Having Imino Group as Linking Group and Having Hydrolyzable Silyl Group and/or Silanol Group>

The synthesis method for Component C having an imino group as a linking group (hereinafter, appropriately referred to as “imino linking group-containing Component C”) is not particularly limited; however, specific examples thereof include synthesis methods such as a reaction between Component C having an amino group and a halogenated hydrocarbon; a reaction between Component C having an amino group and Component C having a halogenated hydrocarbon group; a reaction between Component C having a halogenated hydrocarbon group and an amine; a reaction between Component C having an amino group and an oxirane; a reaction between Component C having an amino group and Component C having an oxirane group; a reaction between an amine and Component C having an oxirane group; a reaction between Component C having an amino group and an aziridine; a reaction between Component C having an ethylenically unsaturated double bond and an amine; a reaction between Component C having an ethylenically unsaturated double bond and Component C having an amino group; a reaction between a compound having an ethylenically unsaturated double bond and Component C having an amino group; a reaction between a compound having an acetylenic unsaturated triple bond and Component C having an amino group; a reaction between Component C having an iminic unsaturated double bond and an organic alkali metal compound; a reaction between Component C having an iminic unsaturated double bond and an organic alkaline earth metal compound; and a reaction between a carbonyl compound and Component C having an amino group.

<Synthesis Method for Compound Having Ureylene Group as Linking Group and Having Hydrolyzable Silyl Group and/or Silanol Group>

The synthesis method for Component C having a ureylene group as a linking group (hereinafter, appropriately referred to as “ureylene linking group-containing Component C”) is not particularly limited; however, specific examples thereof include synthesis methods such as a reaction between Component C having an amino group and an isocyanic acid ester; a reaction between Component C having an amino group and Component having an isocyanic acid ester; and a reaction between an amine and Component C having an isocyanic acid ester.

Component C is preferably a compound represented by the following Formula (C-1) or Formula (C-2).

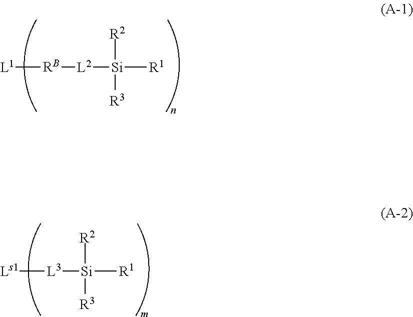

(In Formula (C-1) and Formula (C-2), R^{B} represents an ester bond, an amide bond, a urethane bond, a urea bond, or an imino group; L¹ represents an n-valent linking group; L² represents a divalent linking group; L^{s1} represents an m-valent linking group; L³ represents a divalent linking group; n and m each independently represent an integer of 1 or larger; and R¹ to R³ each independently represent a hydrogen atom, a halogen atom, or a monovalent organic substituent; provided that at least any one of R¹ to R³ represents a hydrolyzable group selected from the group consisting of an alkoxy group, a mercapto group, a halogen atom, an amide group, an acetoxy group, an amino group and an isopropenoxy group, or a hydroxyl group.)

R¹ to R³ in the above Formula (C-1) and Formula (C-2) have the same meanings as R¹ to R¹ in the above Formula (1), respectively, and preferred ranges thereof are also the same.

R^{B} is preferably an ester bond or a urethane bond, and more preferably an ester bond, from the viewpoints of rinsability and the film strength.

The divalent or n-valent linking group for L¹ to L³ is preferably a group composed of at least one kind of atom selected from the group consisting of a carbon atom, a hydrogen atom, an oxygen atom, a nitrogen atom and a sulfur atom, and is more preferably a group composed of at least one kind of atom selected from the group consisting of a carbon atom, a hydrogen atom, an oxygen atom and a sulfur atom. The number of carbon atoms of L¹ to L³ is preferably 2 to 60, and more preferably 2 to 30.

The m-valent linking group for L^{s1} is preferably a group composed of at least one kind of atom selected from the group consisting of a sulfur atom, a carbon atom, a hydrogen atom, an oxygen atom, a nitrogen atom and a sulfur atom, and more preferably an alkylene group, or a group combining two or more of an alkylene group, a sulfide group and an imino group. The number of carbon atoms of L^{s1} is preferably 2 to 60, and more preferably 6 to 30.

n and m are each independently preferably an integer from 1 to 10, more preferably an integer from 2 to 10, even more preferably an integer from 2 to 6, and particularly preferably 2.

The n-valent linking group for L¹ and/or the divalent linking group for L², or the divalent linking group for L³ preferably has an ether bond, and more preferably has an ether bond that is contained in an oxyalkylene group, from the viewpoint of the removability (rinsability) of the engraving residue.

Among the compounds represented by Formula (C-1) or Formula (C-2), from the viewpoint of crosslinkability or the like, the n-valent linking group for L¹ and/or the divalent linking group for L² in Formula (C-1) is a group having a sulfur atom.

Specific examples of Component C that can be applied to the present invention are listed below. Examples include vinyltrichlorosilane, vinyltrimethoxysilane, vinyltriethoxysilane, β-(3,4-epoxycyclohexyl)ethyltrimethoxysilane, γ-glycidoxypropyltrimethoxysilane, γ-glycidoxypropylmethyldiethoxysilane, γ-glycidoxypropyltriethoxysilane, γ-methacryloxypropylmethyldimethoxysilane, p-styryltrimethoxysilane, γ-methacryloxypropyltrimethoxysilane, γ-methacryloxypropylmethyldiethoxysilane, γ-methacryloxypropyltriethoxysilane, γ-acryloxypropyltrimethoxysilane, N-(β-aminoethyl)-γ-aminopropylmethyldimethoxysilane, N-(β-aminoethyl)-γ-aminopropyltrimethoxysilane, N-(β-aminoethyl)-γ-aminopropyltriethoxysilane, γ-aminopropyltrimethoxysilane, γ-aminopropyltriethoxysilane, N-phenyl-γ-aminopropyltrimethoxysilane, γ-mercaptopropyltrimethoxysilane, γ-mercaptopropyltriethoxysilane, mercaptomethyltrimethoxysilane, dimethoxy-3-mercaptopropylmethylsilane, 2-(2-aminoethylthioethyl)diethoxy methylsilane, 3-(2-acetoxyethylthiopropyl)dimethoxymethylsilane, 2-(2-aminoethylthioethyl)triethoxysilane, dimethoxymethyl-3-(3-phenoxypropylthiopropyl)silane, bis(triethoxysilylpropyl) disulfide, bis(triethoxysilylpropyl) tetrasulfide, 1,4-bis(triethoxysilyl)benzene, bis(triethoxysilyl)ethane, 1,6-bis(trimethoxysilyl)hexane, 1,8-bis(triethoxysilyl)octane, 1,2-bis(trimethoxysilyl)decane, bis(triethoxysilylpropyl)amine, bis(trimethoxysilylpropyl)urea, γ-chloropropyltrimethoxysilane, γ-ureidopropyltriethoxysilane, trimethylsilanol, diphenylsilanediol, and triphenylsilanol. In addition to these, the compounds shown below may be mentioned as preferred examples; however, the present invention is not intended to be limited to these compounds.

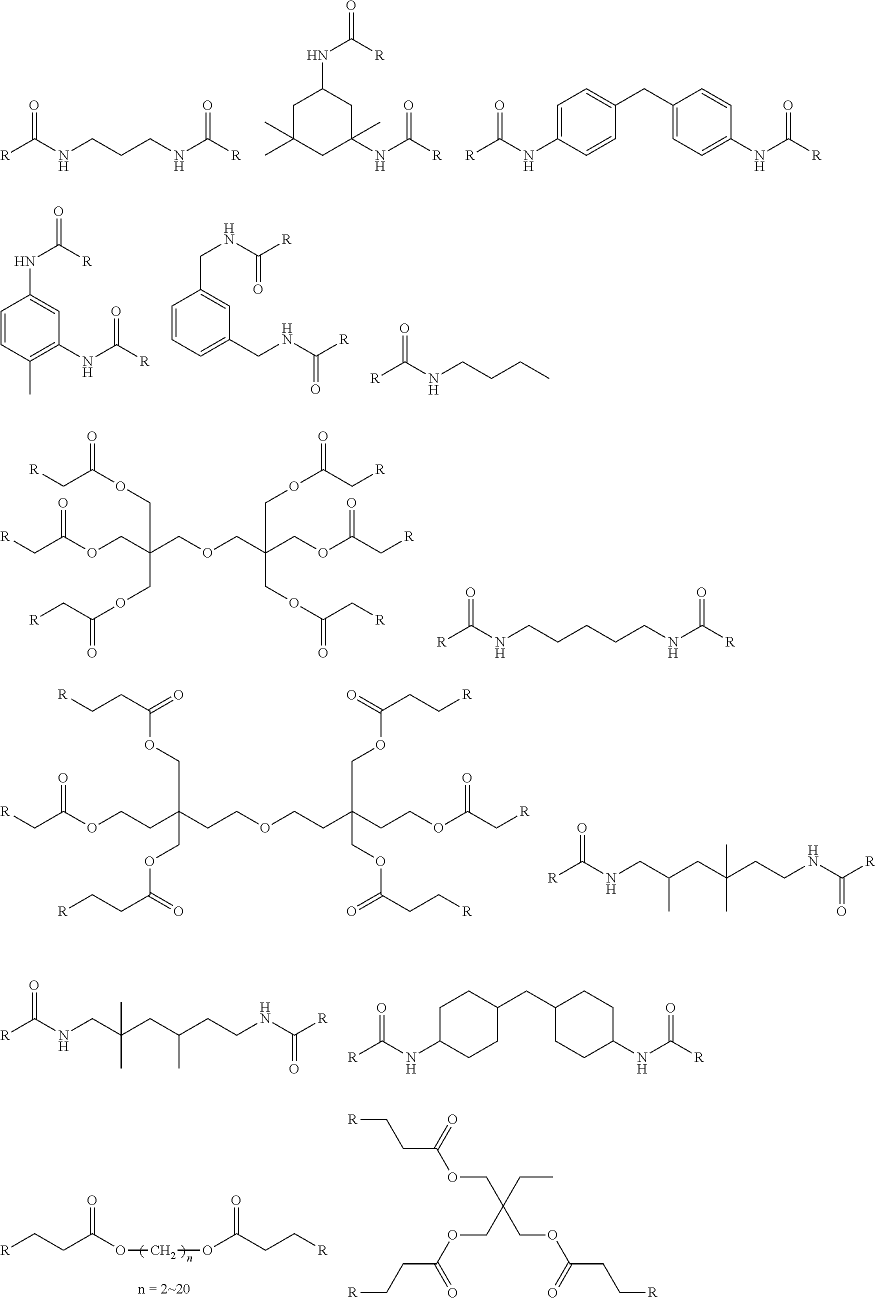

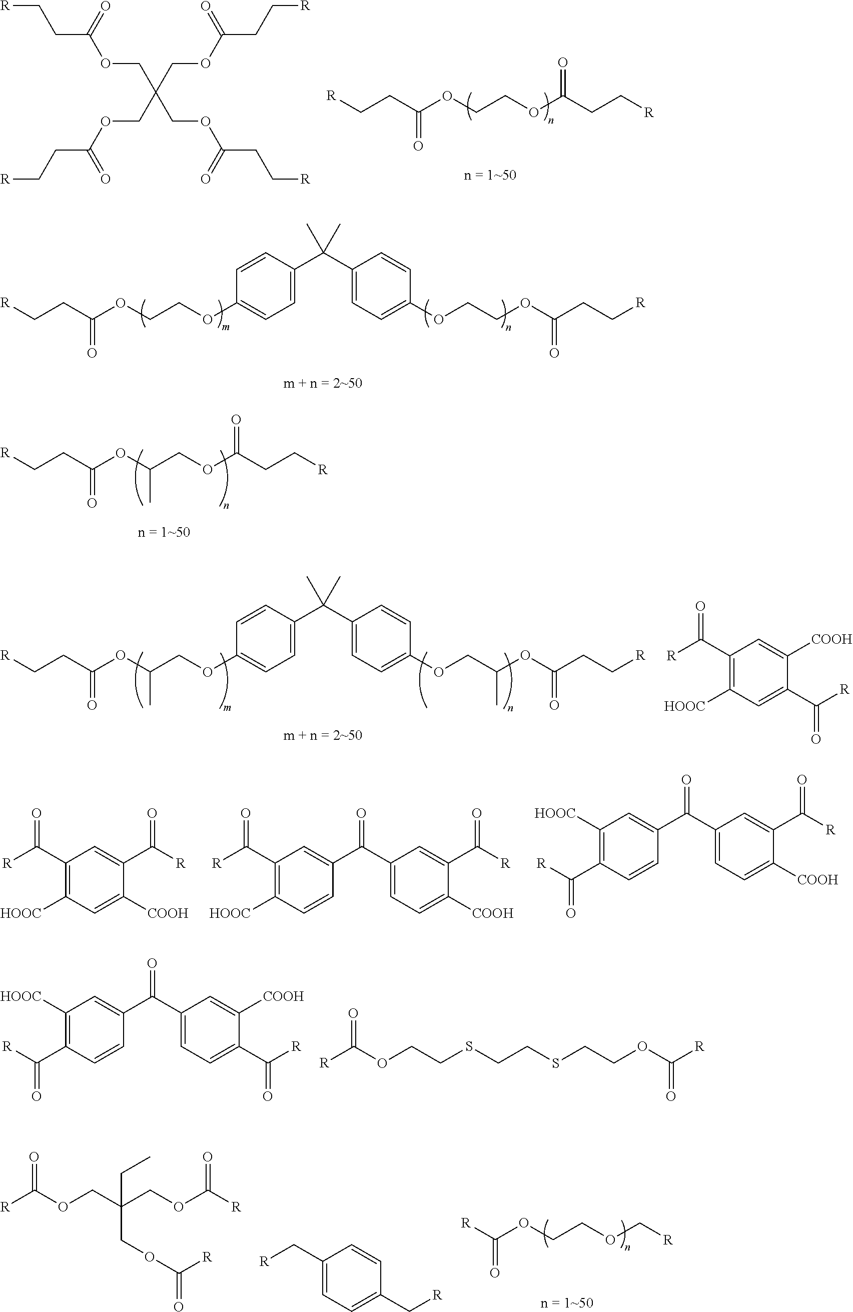

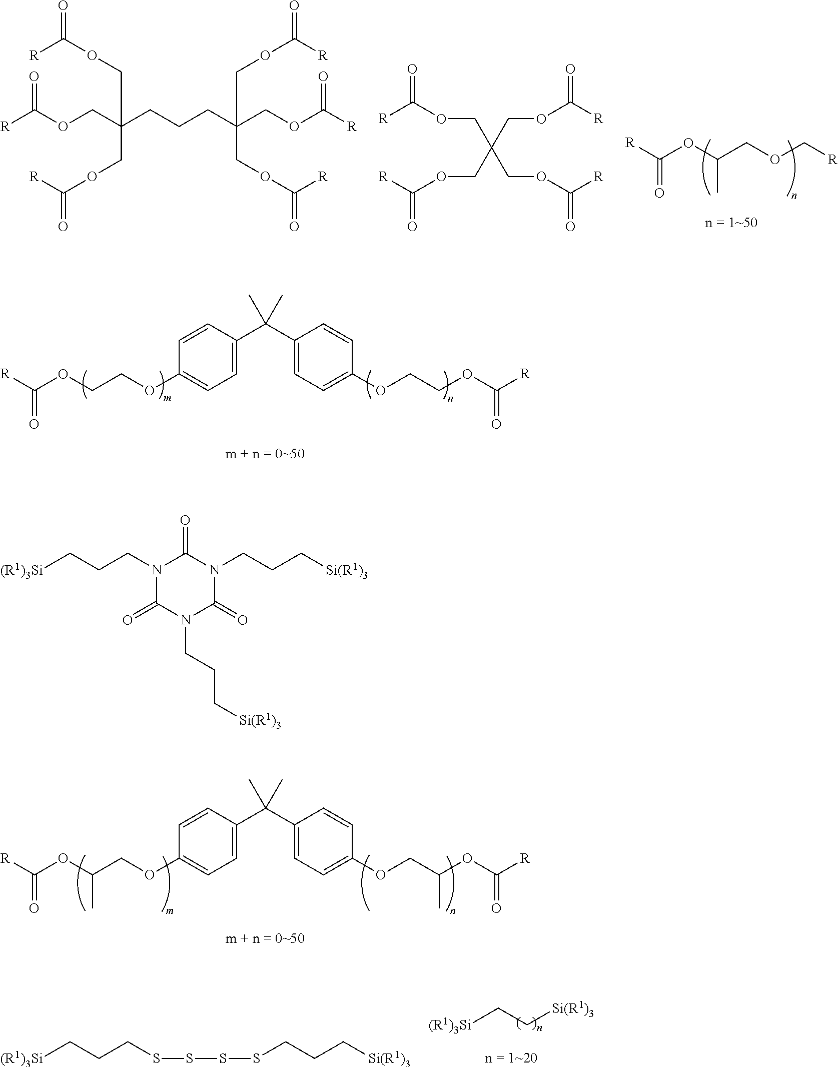

In the respective formulae described above, R represents a partial structure selected from the following structures. In a case in which plural R's and plural R¹'s are present in the molecule, these may be identical to or different from each other, and in view of the suitability for synthesis, it is preferable that these groups are identical.

### R:

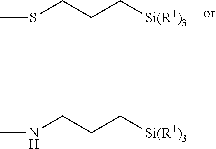

R¹: —OCH₃, —OCH₂CH₃,

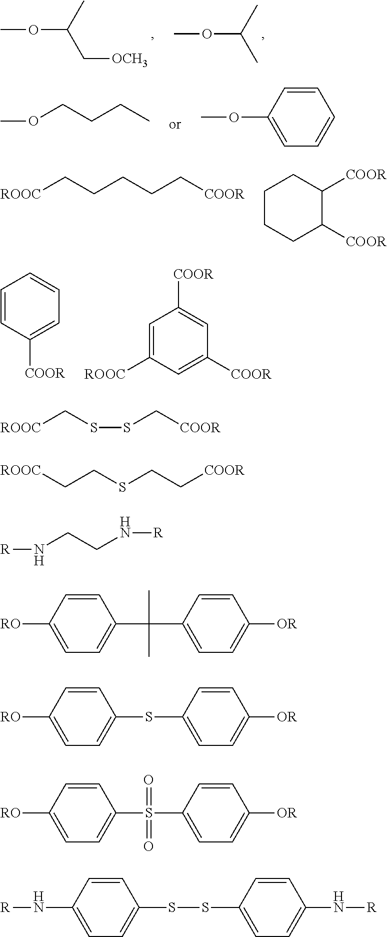

In the respective formulae described above, R represents a partial structure shown below. R¹ has the same meaning as R¹ described above. In a case in which plural R's and plural R¹'s are present in the molecule, these may be identical to or different from each other, and in view of the suitability for synthesis, it is preferable that these groups are identical.

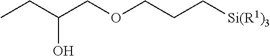

Component C can be obtained by appropriately synthesizing the compound; however, it is preferable to use a commercially available product from the viewpoint of cost. Regarding Component C, for example, commercially available products such as manufactured silane products and silane coupling agents marketed from Shin-Etsu Chemical Co., Ltd., Dow Corning Toray Co., Ltd., Momentive Performance Materials, Inc., Chisso Corporation., and the like, correspond to this Component C, and therefore, these commercially available products may be appropriately selected and used according to the purpose in the resin composition of the invention.

As Component C according to the invention, a partial hydrolytic condensate obtained using one compound having a hydrolyzable silyl group and/or a silanol group, or a partial co-hydrolytic condensate obtained using two or more kinds of such a compound, can be used. Hereinafter, these compounds may be referred to as “partial (co)hydrolytic condensates”.

Among the silane compounds as partial (co)hydrolyzable condensate precursors, from the viewpoints of general-purpose usability, cost, and compatibility of the film, a silane compound having a substituent selected from a methyl group and a phenyl group as a substituent on silicon is preferred, and specifically, examples of preferred precursors include methyltrimethoxysilane, methyltriethoxysilane, phenyltrimethoxysilane, phenyltriethoxysilane, dimethyldimethoxysilane, dimethyldiethoxysilane, diphenyldimethoxysilane, and diphenyldiethoxysilane.

In this case, as the partial (co)hydrolytic condensate, a dimer of a silane compound such as described above (a product obtained by subjecting 2 moles of the silane compound to 1 mole of water to thereby release 2 moles of alcohol, and converting the silane compound to a disiloxane unit) to a 100-mer, preferably a dimer to a 50-mer., and more preferably a dimer to a 30-mer of the silane compound can be suitably used, and it is also possible to use a partial co-hydrolytic condensate obtained from two or more kinds of silane compounds as raw materials.

Meanwhile, regarding such a partial (co)hydrolytic condensate, a product commercially available as a silicone alkoxy oligomer may be used (for example, commercially available from Shin-Etsu Chemical Co., Ltd.), and, based on a conventional method, a product produced by reacting a hydrolyzable silane compound with water of hydrolysis in an amount less than the equivalent amount, and then removing side products such as alcohol and hydrochloric acid, may also be used. On the occasion of production, in a case in which, for example, an alkoxysilane or an acyloxysilane such as described above is used as the hydrolyzable silane compound as a raw material for obtaining a precursor, partial hydrolytic condensation may be carried out using an acid such as hydrochloric acid or sulfuric acid; a hydroxide of an alkali metal or an alkaline earth metal, such as sodium hydroxide or potassium hydroxide; an alkaline organic substance such as triethylamine or the like as a reaction catalyst. In a case in which the hydrolyzable silane compound is directly produced from a chlorosilane, water and an alcohol may be reacted using hydrochloric acid that is produced as a side product, as a catalyst.

It is preferable that the resin composition of the invention includes, if necessary, optional components such as a polymerizable monomer, a polymerization initiator, a vulcanization accelerating agent, and a photothermal conversion agent, together with Component A and Component B as essential components according to the invention, and Component C as an optional component.

These optional components (Component D) to (Component G) will be described in detail below.

(Component D) Polymerizable Monomer

It is preferable that the resin composition for laser engraving of the invention includes (Component D) a polymerizable monomer, from the viewpoint of increasing the rupture strength of the relief layer.

Component D used for the invention is not particularly limited, and any known polymerizable monomer can be used; however, Component D is preferably a radical polymerizable monomer. Furthermore, Component D is preferably a polyfunctional ethylenically unsaturated compound having two or more radical polymerizable groups in the molecule. The number of radical polymerizable groups carried by Component D is preferably 2 to 20, more preferably 2 to 6, and particularly preferably 2.

Component D is preferably a polymerizable compound having a molecular weight of less than 2,000, and more preferably a polymerizable compound having a (weight average) molecular weight of 170 to 1.000.

Such polyfunctional ethylenically unsaturated compounds are widely known in the relevant industrial field, and these compounds can be used without any particular limitations in the present invention. Examples of Component D include a carboxylic acid having an ethylenically unsaturated group; an ester obtainable by a reaction between a polyhydric alcohol (polyol) and a carboxylic acid (derivative) having an ethylenically unsaturated group; an amide obtainable by a reaction between a polyvalent amine (polyamine) and a carboxylic acid having an ethylenically unsaturated group; a polyfunctional vinyl ether; and a polyfunctional allyl compound. These will be explained in detail below.

Examples of the polyfunctional ethylenically unsaturated compound include unsaturated carboxylic acids (for example, acrylic acid, methacrylic acid, itaconic acid, crotonic acid, isocrotonic acid, and maleic acid), esters thereof, and amides thereof.

Preferably, an ester between an unsaturated carboxylic acid and an aliphatic polyhydric alcohol compound, or an amide between an unsaturated carboxylic acid and an aliphatic polyvalent amine compound is used.

Preferred examples of the aliphatic polyhydric alcohol include an alkylenediol having 2 to 10 carbon atoms, trimethylolpropane, pentaerythritol, dipentaerythritol, and tricyclodecanedimethanol.

Furthermore, an addition reaction product between an unsaturated carboxylic acid ester or amide having a nucleophilic substituent such as a hydroxyl group or an amino group, and a polyfunctional isocyanate or an epoxy compound; a dehydration condensation reaction product between such an unsaturated carboxylic acid ester or amide and a polyfunctional carboxylic acid; and the like are also suitably used. Furthermore, suitable examples also include an addition reaction product between an unsaturated carboxylic acid ester or amide and a monofunctional or polyfunctional alcohol or amine, which has an electrophilic substituent such as an isocyanate group or an epoxy group; and a substitution reaction product between an unsaturated carboxylic acid ester or amide and a monofunctional or polyfunctional alcohol or amine, which has a leaving substituent such as a halogen atom or a tosyloxy group. As other examples, a group of compounds obtained by replacing the unsaturated carboxylic acid in the above-described compounds with a vinyl compound, an allyl compound, an unsaturated phosphonic acid, styrene or the like can also be used.

The radical polymerizable group contained in the polyfunctional ethylenically unsaturated compound is preferably a (meth)acryloyl group, and more preferably a (meth)acryloyloxy group, from the viewpoint of reactivity.

Specific examples of a monomer of an ester between an aliphatic polyhydric alcohol compound and an unsaturated carboxylic acid (hereinafter, also referred to as “ester monomer”) include, as acrylic acid esters, ethylene glycol diacrylate, triethylene glycol diacrylate, 1,3-butanediol diacrylate, tetramethylene glycol diacrylate, propylene glycol diacrylate, neopentyl glycol diacrylate, trimethylolpropane triacrylate, trimethylolpropane tri(acryloyloxypropyl) ether, trimethylolethane triacrylate, hexanediol diacrylate, 1,4-cyclohexanediol diacrylate, tetraethylene glycol diacrylate, pentaerythritol diacrylate, pentaerythritol triacrylate, pentaerythritol tetraacrylate, dipentaerythritol diacylate, dipentaerythritol hexaacrylate, sorbitol triacrylate, sorbitol tetraacrylate, sorbitol pentaacrylate, sorbitol hexaacrylate, tri(acryloyloxyethyl) isocyanurate, and polyester acrylate oligomers.

Examples of a methacrylic acid ester include tetramethylene glycol dimethacrylate, triethylene glycol dimethacrylate, neopentyl glycol dimethacrylate, trimethylolpropane trimethacrylate, trimethylolethane trimethacrylate, ethylene glycol dimethacrylate, 1,3-butanediol dimethacrylate, hexanediol dimethacrylate, pentaerythritol dimethacrylate, pentaerythritol trimethacrylate, pentaerythritol tetramethacrylate, dipentaerythritol dimethacrylate, dipentaerythritol hexamethacrylate, sorbitol trimethacrylate, sorbitol tetramethacrylate, bis[p-(3-methacryloxy-2-hydroxypropoxy)phenyl]dimethylmethane, and bis[p-(methacryloxyethoxy)phenyl]dimethylmethane.

Examples of an itaconic acid ester include ethylene glycol diitaconate, propylene glycol diitaconate, 1,3-butanediol diitaconate, 1,4-butanediol diitaconate, tetramethylene glycol diitaconate, pentaerythritol diitaconate, and sorbitol tetraitaconate.

Examples of a crotonic acid ester include ethylene glycol dicrotonate, tetramethylene glycol dicrotonate, pentaerythritol dicrotonate, and sorbitol tetracrotonate.

Examples of an isocrotonic acid ester include ethylene glycol diisocrotonate, pentaerythritol diisocrotonate, and sorbitol tetraisocrotonate.

Examples of a maleic acid ester include ethylene glycol dimaleate, triethylene glycol dimaleate, pentaerythritol dimaleate, and sorbitol tetramaleate.

As examples of other esters, for example, the aliphatic alcohol-based esters described in JP1971-27926B (JP-S46-27926B), JP1976-47334B (JP-S51-47334B), and JP1982-196231A (JP-S57-196231A); esters having an aromatic skeleton as described in JP1984-5240A (JP-S59-5240A), JP1984-5241A (JP-S59-5241A), and JP1990-226149A (JP-H02-226149A); and esters containing an amino group as described in JP1989-165613A (JP-H01-165613A) are also suitably used.

The ester monomers described above can also be used as mixtures.

Furthermore, specific examples of an amide between an aliphatic polyvalent amine compound and an unsaturated carboxylic acid include methylene bisacrylamide, methylene bismethacrylamide, 1,6-hexamethylene bisacrylamide, 1,6-hexamethylene bismethacrylamide, diethylenetriamine trisacrylamide, xylylene bisacrylamide, and xylylenebismethacrylamide.

Furthermore, a urethane-based addition polymerizable compound produced using an addition reaction between an isocyanate and a hydroxyl group is also suitable, and specific examples thereof include, for example, a polyfunctional ethylenically unsaturated compound obtained by adding an ethylenically unsaturated compound containing a hydroxyl group, which is represented by the following Formula (1), to a polyisocyanate compound having two or more isocyanate groups in one molecule, as described in JP1973-41708B (JP-S48-41708B).

CH₂═C(R)COOCH₂CH(R′)OH (1)
(provided that R and R′ each represent H or CH₃.)

Furthermore, the urethane acrylates described in JP1976-37193A (JP-S51-37193A), JP1990-32293B (JP-H02-32293B), and JP1990-16765B (JP-H02-16765B); and urethane compounds having an ethylene oxide-based skeleton as described in JP1983-49860B (JP-S58-49860B), JP1981-17654B (JP-S56-17654B), JP1987-39417B (JP-S62-39417B), and JP1987-39418B (JP-S62-39418B) are also suitable.

Furthermore, a curable composition can be obtained in a short period of time by using the addition polymerizable compounds having an amino structure in the molecule as described in JP1988-277653A (JP-S63-277653A), JP1988-260909A (JP-S63-260909A), and JP1989-105238A (JP-H01-105238A).

Other examples include polyfunctional acrylates or methacrylates such as the polyester acrylates described in JP1973-64183A (JP-S48-64183A), JP1974-43191B (JP-S49-43191B), and JP1977-30490B (JP-S52-30490B); and epoxy acrylates obtained by reacting epoxy resins with (meth)acrylic acid. Further examples include the particular unsaturated compounds described in JP1971-43946B (JP-S46-43946B), JP1989-40337B (JP-H01-40337B), and JP1989-40336B (JP-H01-40336B); and the vinylphosphonic acid-based compounds described in JP1990-25493A (JP-H02-25493A). In a certain case, the structure containing a perfluoroalkyl group described in JP1986-22048A (JP-S61-22048A) is suitably used. The compounds introduced as photocurable monomers and oligomers in Journal of the Adhesion Society of Japan, Vol. 20, No. 7, pp. 300-308 (1984) can also be used.

Examples of the vinyl compound include butanediol-1,4-divinyl ether, ethylene glycol divinyl ether, 1,2-propanediol divinyl ether, 1,3-propanediol divinyl ether, 1,3-butanediol divinyl ether, 1,4-butanediol divinyl ether, neopentyl glycol divinyl ether, trimethylolpropane trivinyl ether, trimethylolethane trivinyl ether, hexanediol divinyl ether, tetraethylene glycol divinyl ether, pentaerythritol divinyl ether, pentaerythritol trivinyl ether, pentaerythritol tetravinyl ether, sorbitol tetravinyl ether, sorbitol pentavinyl ether, ethylene glycol diethylene vinyl ether, ethylene glycol dipropylene vinyl ether, trimethylolpropane triethylene vinyl ether, trimethylolpropane diethylene vinyl ether, pentaerythritol diethylene vinyl ether, pentaerythritol triethylene vinyl ether, pentaerythritol tetraethylene vinyl ether, 1,1,1-tris[4-(2-vinyloxyethoxy)phenyl]ethane, bisphenol A divinyloxyethyl ether, and divinyl adipate.

Examples of the allyl compound include polyethylene glycol diallyl ether, 1,4-cyclohexane diallyl ether, 1,4-diethylcyclohexyl diallyl ether, 1,8-octane diallyl ether, trimethylolpropane diallyl ether, trimethylolethane triallyl ether, pentaerythritol triallyl ether, pentaerythritol tetraallyl ether, dipentaerythritol pentaallyl ether, dipentaerythritol hexaallyl ether, diallyl phthalate, diallyl terephthalate, diallyl isophthalate, triallyl isocyanurate, and triallyl phosphate.

Among these, Component D is preferably an ester between an aliphatic polyhydric alcohol compound and (meth)acrylic acid, and more preferred examples include diethylene glycol di(meth)acrylate, dipentaerythritol hexa(meth)acrylate, tricyclodecane dimethanol di(meth)ary late, trimethylolpropane tri(meth)acrylate, pentaerythritol tetra(meth)acrylate, and 1,6-hexanediol di(meth)acrylate.

Furthermore, regarding Component D, commercially available products can be used, and examples thereof hexanediol diacrylate (manufactured by Dai-ichi Kogyo Seiyaku Co., Ltd.) and trimethylolpropane triacrylate (manufactured by Shin Nakamura Chemical Co., Ltd.).

Component D may be used singly, or two or more kinds thereof may be used in combination.

The content of Component D is preferably 3% by mass to 20% by mass, and more preferably 5% by mass to 15% by mass, relative to the total solid content mass of the resin composition for laser engraving.

(Component E) Polymerization Initiator

The resin composition for laser engraving of the invention may include (Component E) a polymerization initiator in order to accelerate the formation of a crosslinked structure.

Regarding the polymerization initiator of the invention, any polymerization initiator that is known to those ordinarily skilled in the art can be used without any limitations; however, the polymerization initiator is preferably a radical polymerization initiator. Furthermore, any of a thermal polymerization initiator or a photopolymerization initiator can be used; however, it is preferable to use a thermal polymerization initiator. Detailed descriptions will be given below; however, the present invention is not intended to be limited by these descriptions.

According to the invention, preferred examples of a radical polymerization initiator include: (a) an aromatic ketone, (b) an onium salt compound, (c) an organic peroxide, (d) a thio compound, (e) a hexaarylbiimidazole compound, (f) a keto oxime ester compound, (g) a borate compound, (h) an azinium compound, (i) a metallocene compound, (j) an active ester compound, (k) a compound having a carbon-halogen bond, and (1) an azo-based compound. Furthermore, regarding the items (a) to (1), the compounds listed in paragraphs “0074” to “0118” of JP 2008-63554A can be preferably used. Specific examples of the above items (a) to (1) will be listed below; however, the present invention is not intended to be limited to these.

According to the invention, from the viewpoint of the engraving sensitivity and from the viewpoint that when the resin composition is applied to a relief forming layer of a flexographic printing plate precursor, the relief edge shape is made satisfactory, the (a) aromatic ketone, the (c) organic peroxide, and the (1) azo-based compound are more preferred, and the (a) aromatic ketone and the (c) organic peroxide are even more preferred, while the (c) organic peroxide is particularly preferred.

Furthermore, regarding the (a) aromatic ketone, (c) organic peroxide, and (1) azo-based compound, the compounds shown below are preferred.

(a) Aromatic Ketone

Regarding the (a) aromatic ketone that is preferable as the radical polymerization initiator that can be used for the invention, benzophenone-based or acetophenone-based compounds such as benzophenone, 4,4′-(bisdimethylamino)benzophenone, 4,4′-(bisdiethylamino)benzophenone, 4,4′-dichlorobenzophenone, 1-hydroxycyclohexyl phenyl ketone, 2-benzyl-2-dimethylamino-1-(4-morpholinophenyl)butanone-1, 2-tolyl-2-dimethylamino-1-(4-morpholinophenyl)butanone-1, and 2-methyl-1-[4-(methylthio)phenyl]-2-morpholino-I-propane are preferred, and acetophenone-based compounds are more preferred.

(c) Organic Peroxide

Regarding the (c) organic peroxide that is preferable as the radical polymerization initiator that can be used for the invention, peroxyester-based compounds such as 3,3′,4,4′-tetra(t-butylperoxycarbonyl)benzophenone, 3,3′,4,4′-tetra(t-amylperoxycarbonyl)benzophenone, 3.3′,4,4′-tetra(t-hexylperoxycarbonyl)benzophenone, 3,3′,4,4′-tetra(t-octylperoxycarbonyl)benzophenone, 3,3′,4,4′-tetra(cumylperoxycarbonyl)benzophenone, 3,3′,4,4′-tetra(p-isopropylcumylperoxycarbonyl)benzophenone, di-t-butyl diperoxyisophthalate, t-butyl peroxybenzoate, t-butyl peroxy-3-methylbenzoate, t-butyl peroxylaurate, t-butyl peroxypivalate, t-butyl peroxy-2-ethylhexanoate, t-butyl peroxy-3,5,5-trimethylhexanoate, t-butyl peroxyneoheptanoate, t-butyl peroxyneodecanoate, and t-butyl peroxyacetate; α,α′-di(t-butylperoxy)diisopropylbenzene, t-butyl cumyl peroxide, di-t-butyl peroxide, t-butyl peroxyisopropyl monocarbonate, and t-butyl peroxy-2-ethylhexyl monocarbonate are preferred, and peroxyester-based organic peroxides are more preferred, while t-butyl peroxy-2-ethylhexyl monocarbonate is even more preferred.

(1) Azo-Based Compound

Examples of the (1) azo-based compound that is preferable as the radical polymerization initiator that can be used for the invention include 2,2′-azobisisobutyronitrile, 2,2′-azobispropionitrile, 1,1′-azobis(cyclohexane-1-carbonitrile), 2,2′-azobis(2-methylbutyronitrile), 2,2′-azobis(2,4-dimethylvaleronitrile), 2,2′-azobis(4-methoxy-2,4-dimethylvaleronitrile), 4,4′-azobis(4-cyanovaleric acid), dimethyl 2,2′-azobisisobutyrate, 2,2′-azobis(2-methylpropionamideoxime), 2,2′-azobis[2-(2-imidazolin-2-yl)propane], 2,2′-azobis {2-methyl-N-[1,1-bis(hydroxymethyl)-2-hydroxyethyl]propionamide}, 2,2′-azobis[2-methyl-N-(2-hydroxyethyl)propionamide], 2,2′-azobis(N-butyl-2-methylpropionamide), 2,2′-azobis(N-cyclohexyl-2-methylpropionamide), 2,2′-azobis[N-(2-propenyl)-2-methylpropionamide], and 2,2′-azobis(2,4,4-trimethylpentane).

Meanwhile, regarding the polymerization initiator according to the invention, the (c) organic peroxide is preferred from the viewpoint of crosslinkability of the resin composition, and as an unexpected effect, the (c) organic peroxide is particularly preferable from the viewpoint of enhancing the engraving sensitivity.

According to the invention, the radical polymerization initiator may be used singly, or in combination of two or more kinds thereof.

According to the invention, the content of Component E in the resin composition for laser engraving is preferably 0.1% by mass to 10% by mass, more preferably 0.5% by mass to 5% by mass, and even more preferably 0.8% by mass to 3% by mass, relative to the total solid content mass of the resin composition.

Furthermore, regarding Component E, a commercially available product can also be used, and examples include PERBUTYL E (manufactured by NOF Corporation.)

(Component F) Vulcanization Accelerating Agent

It is preferable that the resin composition of the invention includes (Component F) a vulcanization accelerating agent, for the purpose of accelerating a vulcanization (crosslinking) reaction of Component A by Component B, or of regulating the degree of vulcanization.

The vulcanization accelerating agent according to the invention is not particularly limited, and examples thereof include guanidine-based compounds such as diphenylguanidine; thiuram-based compounds such as tetramethylthiuram disulfide, tetramethylthiuram monosulfide, tetraethylthiuram disulfide, and tetrabutylthiuram disulfide; dithiocarbamic acid salt-based compounds such as zinc dimethyldithiocarbamate; thiazole-based compounds such as 2-mercaptobenzothiazole and dibenzothiazyl disulfide; and sulfenamide-based compounds such as N-cyclohexyl-2-benzothiazole sulfenamide and N-t-butyl-2-benzothiazole sulfenamide. Among them, the vulcanization accelerating agent is preferably a thiazole-based or sulfenamide-based vulcanization accelerating agent.

It is preferable that the resin composition of the invention includes Component F in an amount of 0.1 parts by mass to 10 parts by mass, and more preferably 0.5 parts by mass to 5 parts by mass, relative to 100 parts by mass of Component A.

(Component G) Photothermal Conversion Agent

It is preferable that the resin composition of the invention further includes (Component G) a photothermal conversion agent, and it is more preferable that the photothermal conversion agent is capable of absorbing light having a wavelength of 700 nm to 1.300 nm. That is, the photothermal conversion agent according to the invention is considered to accelerate thermal decomposition of a cured product at the time of laser engraving, by absorbing laser light and generating heat. Accordingly, it is preferable to select a photothermal conversion agent which absorbs light having the wavelength of the laser used for engraving.

In a case in which the crosslinked relief forming layer in the flexographic printing plate precursor for laser engraving of the invention is used for laser engraving by using a laser which emits infrared radiation at 700 nm to 1,300 nm (a YAG laser, a semiconductor laser, a fiber laser, a surface emitting laser, or the like) as a light source, for the photothermal conversion agent, it is preferable to use a compound having a maximum absorption wavelength of 700 nm to 1,300 nm.

Examples of the photothermal conversion agent that can be used for the invention include various dyes and pigments.

Among the photothermal conversion agents, regarding dyes, commercially available dyes and known dyes described in the literature such as, for example, “Senryo Benran (Handbook of Dyes)” (edited by the Society of Organic Synthesis Chemistry, Japan, published in 1970) can be utilized. Specifically, a photothermal conversion agent having a maximum absorption wavelength of 700 nm to 1,300 nm is preferably used, and examples thereof include dyes such as an azo dye, a metal complex salt azo dye, a pyrazolone azo dye, a naphthoquinone dye, an anthraquinone dye, a phthalocyanine dye, a carbonium dye, a diimmonium compound, a quinoneimine dye, a methine dye, a cyanine dye, a squarylium coloring matter, a pyrylium salt, and a metal thiolate complex.

Examples of the dye that is preferably used for the invention include cyanine-based coloring matters such as a heptamethine cyanine coloring matter; oxonol-based coloring matters such as a pentamethine oxonol coloring matter; phthalocyanine-based coloring matters; and the dyes described in paragraphs “0124” to “0137” of JP2008-63554A.

Among the photothermal conversion agents used for the invention, regarding the pigment, commercially available pigments and those pigments described in the Handbook of Color Index (C.I.). “Saishin Ganryo Benran (Latest Pigment Handbook)” (edited by Japan Society of Pigment Technologies, published in 1977), “Saishin Ganryo Oyo Gijutsu (Latest Pigment Application and Technologies)” (published by CMC Publishing Co., Ltd., in 1986), and “Insatsu Inki Gijutsu (Printing Ink Technology)” (published by CMC Publishing Co., Ltd. in 1984), can be utilized.

Examples of the kind of the pigment that can be used include a black pigment, a yellow pigment, an orange pigment, a brown pigment, a red pigment, a purple pigment, a blue pigment, a green pigment, a fluorescent pigment, a metal powder pigment, and a polymer-conjugated pigment. Specifically, an insoluble azo pigment, an azo lake pigment, a condensed azo pigment, a chelate azo pigment, a phthalocyanine pigment, an anthraquinone-based pigment, a perylene- and perinone-based pigment, a thioindigo pigment, a quinacridone-based pigment, a dioxazine-based pigment, an isoindolinone-based pigment, a quinophthalone-based pigment, a dyeing lake pigment, an azine pigment, a nitroso pigment, a nitro pigment, a natural pigment, a fluorescent pigment, an inorganic pigment, and carbon black. Among these pigments, preferred is carbon black.

Any carbon black can all be used without being limited by the classification according to ASTM or the use (for example, color application, rubber application, or battery application), as long as dispersibility in the composition and the like are stable. Examples of carbon black include furnace black, thermal black, channel black, lamp black, and acetylene black. Meanwhile, a black colorant such as carbon black can be used in the form of a color chip or a color paste, in which the black colorant has been dispersed in advance in nitrocellulose, a binder or the like, using a dispersant as necessary in order to facilitate dispersion, and such a chip or paste can be easily purchased as a commercially available product.

According to the invention, it is preferable to use a carbon black having a relatively low specific surface area and a relatively low dibutyl phthalate absorption (DBP), or a micronized carbon black having a large specific surface area. Suitable examples of carbon black include PRINTEX (registered trademark) U, PRINTEX (registered trademark) A, SPEZIALSCHWARZ (registered trademark) 4 (manufactured by Degussa AG), and #45L (manufactured by Mitsubishi Chemical Corporation).

The carbon black that can be used for the invention is a carbon black having a DBP oil absorption amount of preferably less than 150 ml/100 g, more preferably 100 ml/100 g or less, and even more preferably 70 ml/100 g or less.

Furthermore, from the viewpoint that the engraving sensitivity is enhanced by efficiently transferring the heat generated by photothermal conversion to neighboring polymer molecules and the like, the carbon black is preferably an electroconductive carbon black having a specific surface area of at least 100 m²/g.

The above-mentioned carbon black may be acidic carbon black or basic carbon black. The carbon black is preferably basic carbon black. A mixture with other binders definitely can also be used.

In the case of using carbon black as a photothermal conversion agent, it is preferable to employ not photo-crosslinking that utilizes UV light or the like, but thermal crosslinking, from the viewpoint of curability of the film. When carbon black is used in combination with an organic peroxide as (Component E) a polymerization initiator, which is a preferred component to be used together as described above, it is more preferable because the engraving sensitivity becomes very high.

In regard to the resin composition for laser engraving of the invention, it is preferable to use a polymerization initiator and a photothermal conversion agent capable of absorbing light having a wavelength of 700 nm to 1,300 nm in combination, and it is particularly preferable to use an organic peroxide as Component C and carbon black in combination. According to the embodiment described above, the polymerization initiator remaining in the crosslinked relief forming layer is decomposed by the heat generated from the photothermal conversion agent at the time of laser engraving, thus decomposition of Component A and the like can be further accelerated, and the engraving sensitivity can be enhanced.

The resin composition for laser engraving of the invention may use only one kind of Component G, or may use two or more kinds thereof in combination.

The content of the (Component G) photothermal conversion agent in the resin composition for laser engraving of the invention may vary greatly depending on the magnitude of the molecular extinction coefficient intrinsic to the molecule of the photothermal conversion agent; however, the content is preferably 2% by mass to 60% by mass, more preferably 5% by mass to 30% by mass, and even more preferably 5% by mass to 10% by mass, relative to the total solid content mass of the resin composition.

<Other Additives>

In the resin composition for laser engraving of the invention, additives other than Component A to Component G described above can be appropriately incorporated to the extent that the effects of the invention are not impaired. Examples of the additives include a wax, a process oil, an organic acid, a metal oxide, a fragrance, an ozone decomposition inhibitor, an aging inhibitor, a thermal polymerization inhibitor, and a colorant, and these may be used singly, or in combination of two or more kinds thereof.

<Process Oil>

In the case of using a process oil, for example, an aromatic process oil, a naphthene-based process oil, or a paraffin-based process oil may be used. The amount of use of the process oil is preferably 1 part by mass to 70 parts by mass relative to 100 parts by mass of Component A.

<Organic Acid and Metal Oxide>

The organic acid can be used as an auxiliary agent for vulcanization acceleration by combining the organic acid as a metal salt with a conventional vulcanizing agent. Examples of the organic acid include stearic acid, oleic acid, and murastic acid. Examples of the metal source that is used together include metal oxides such as zinc oxide (zinc white) and magnesium oxide. Regarding these, it is considered that during a vulcanization process, an organic acid and a metal oxide form a metal salt in rubber, and activation of a vulcanizing agent such as sulfur is accelerated thereby. The amount of addition of the metal oxide for forming such a metal oxide in the system is preferably 0.1 parts by mass to 10 parts by mass, and more preferably 2 parts by mass to 10 parts by mass, relative to 100 parts by mass of Component A.

The amount of addition of the organic acid is preferably 0.1 parts by mass to 5 parts by mass, and more preferably 0.1 parts by mass to 3 parts by mass, relative to 100 parts by mass of Component A.

<Fragrance>

It is preferable that the resin composition for laser engraving of the invention includes a fragrance in order to reduce foul odor. The fragrance is effective for reducing the foul odor generated at the time of production of the flexographic printing plate precursor, or at the time of laser engraving.

When the resin composition for laser engraving of the invention includes a fragrance, the solvent odor that is volatilized when the liquid resin composition applied during the production is dried, can be masked. Also, the unpleasant odor generated at the time of laser engraving, such as amine odor, ketone odor, aldehyde odor, or the stinking burnt odor of resins, can be masked.

Furthermore, since the fragrance is also effective for reducing the foul odor of sulfur, the fragrance is useful for the resin composition of the invention that includes a vulcanizing agent.

Regarding the fragrance, any known fragrance can be appropriately selected and used, and one kind of fragrance can be used singly, or plural fragrances can be used in combination.

It is preferable that the fragrance is appropriately selected depending on the silane compound, the vulcanizing agent, the polymer and the like used in the resin composition as described above, and it is preferable to combine known fragrances and optimize the combination. Examples of the fragrance include the fragrances described in “Gosei Koryo—Kagaku to Shohin Chishiki (Synthetic Fragrances—Chemistry and Product Knowledge)” (written by Motoichi Indo, published by The Chemical Daily Co., Ltd.), “Koryo Kagaku Nyumon (Introduction to Fragrance Chemistry)” (written by Shoji Watanabe, published by Baifukan Co., Ltd.). “Kaori no Hyakka (Encyclopedia of Fragrances)” (edited by Japan Perfumery & Flavoring Association, published by Asakura Publishing Co., Ltd.), and “Koryo Kagaku Soran II (Complete Guide to Fragrance Chemistry) Tanri Koryo/Gosei Koryo/Koryo no Oyo (Isolated Fragrances/Synthetic Fragrances/Applications of Fragrances)” (published by Hirokawa Shoten, Ltd.).

Also, examples of the fragrances that can be used for the invention include the fragrances described in paragraphs “0012” to “0025” of JP2009-203310A.

Among them, it is preferable to use, as the fragrance, a terpene compound such as a terpene-based hydrocarbon, a terpene-based alcohol, an oxide of a terpene, a terpene-based aldehyde, a terpene-based ketone, a terpene-based carboxylic acid, a terpene-based lactone, or a terpene-based carboxylic acid ester; and/or an ester compound such as an aliphatic ester, a furan-based carboxylic acid ester, an aliphatic cyclic carboxylic acid ester, a cyclohexylcarboxylic acid ester, or an aromatic carboxylic acid ester.

Furthermore, it is preferable to use a heat-resistant fragrance as the fragrance according to the invention. When a heat-resistant fragrance is used, the fragrance can mask the foul odor caused by decomposition of resins, by emitting an aroma at the time of laser engraving, and a (crosslinked) relief forming layer and a relief layer, which are capable of long-term storage almost without emitting an aroma at normal temperature, can be obtained.

Here, a heat-resistant fragrance means a fragrance which can mask the foul odor caused by decomposition of resins or the like, by emitting an aroma at the time of a laser engraving operation, and is capable of long-term storage almost without emitting an aroma at normal temperature.

Regarding the heat-resistant fragrance, specifically, as shown below, one or more fragrance components selected from the group consisting of heliotrope-based fragrances, jasmine-based fragrances, rose-based fragrances, orange flower-based fragrances, amber-based fragrances, and musk-based fragrances, are preferably used.

Furthermore, more specific examples of the fragrance include TABU type fragrances obtained by adding the below-described fragrance components selected from the group consisting of rose-based fragrances, amber-based fragrances, musk-based fragrances and jasmine-based fragrances, together with solvent dioctyl phthalate (DOP), to the below-described oriental bases containing patchouli oil as a main component.

Oriental bases: patchouli oil, Hercolyn (methyl abietate), vanillin, ethylvanillin, and coumarin

Rose-based fragrance components: phenylethyl alcohol, geraniol, isobornylmethoxycyclohexanol

Amber-based fragrance components: tetrahydro-para-methylquinoline

Musk-based fragrance components: galaxolide and musk ketone

Jasmine-based fragrance components: α-amylcinnamaldehyde and methyl dihydrojasmonate

Furthermore, AMETHYST type fragrances containing the below-described heliotrope-based fragrance components as main fragrance toners, to which jasmine-based fragrance components, and in order to impart high tonality and diffusibility, rose-based fragrance components or orange flower-based fragrance components are added together with solvent DOP, are also preferably used.

Heliotrope-based fragrance components: heliotropine, musk ketone, coumarin, ethylvanillin, acetyl cedrene, Hercolyn (methyl abietate), eugenol, and methylionone Rose-based fragrance components: Damascone-β, Damascone-α, and isobornylmethoxycyclohexanol

Orange flower-based fragrance components: methyl anthranilate, γ-undecalactone, and γ-nonalactone

Jasmine-based fragrance components: methyl dihydrojasmonate

Furthermore, as other heat-resistant fragrances, it is also preferable to use a 6-hydroxyalkanoic acid or a 6-(5- and/or 6-alkenoyloxy)alkanoic acid.

Regarding the fragrance that can be used for the invention, it is preferable that the fragrance includes at least a vanillin-based fragrance, a jasmine-based fragrance, or a mint-based fragrance, and it is more preferable that the fragrance includes a vanillin-based fragrance or a jasmine-based fragrance, while it is even more preferable that the fragrance includes a vanillin-based fragrance.

Furthermore, the fragrance for the resin composition of the invention is preferably a vanillin-based fragrance, a jasmine-based fragrance, or a mint-based fragrance.

Specific preferred examples of the vanillin-based fragrance include vanillin, vanillic acid, vanillyl alcohol, vanillin propylene glycol acetal, methylvanillin, ethylvanillin, para-hydroxybenzoic acid, and para-hydroxybenzaldehyde.

Specific preferred examples of the jasmine-based fragrance include methyl dihydrojasmonate, methyl epidihydrojasmonate, methyl jasmonate, methyl epijasmonate, cis-jasmone, jasmonane, cis-jasmone lactone, dihydrojasmone lactone, jasmine lactone, γ-jasmolactone, cis-jasmone lactone, methyl γ-decalactone, jasmolactone, γ-hexalactone, γ-octalactone, γ-nonalactone, 4-methyl-5-hexenolide-1:4. 2-n-hexylcyclopentanone, and an alkyl-cycloheptylmethyl carbonate.

Specific preferred examples of the mint-based fragrance include menthol, menthone, cineole, l-menthol, d-menthol, dl-menthol, d-neomenthol, d-isomenthol, d-neomenthol, peppermint oil, spearmint oil, and mentha oil.

The content of the fragrance is preferably 0.003% to 1.5% by mass, and more preferably 0.005% to 1.0% by mass, relative to the total solid content mass of the resin composition. When the content is in the above range, the masking effect can be sufficiently exhibited, and the scent of the fragrance is adequate, the operation environment is improved, and the engraving sensitivity becomes excellent.

It is more preferable that nitrocellulose or a high thermal conductive substance is added as an additive for enhancing the engraving sensitivity to the resin composition for laser engraving of the invention.

Since nitrocellulose is an autoreactive compound, the compound itself generates heat at the time of laser engraving, and assists thermal decomposition of the co-existing binder polymer such as a hydrophilic polymer. It is speculated that the engraving sensitivity is enhanced as a result.

A high thermal conductive substance is added for the purpose of assisting heat transfer, and examples of the thermally conductive substance include inorganic compounds such as metal particles, and organic compounds such as an electroconductive polymer. The metal particles are preferably gold microparticles, silver microparticles, and copper microparticles, which have particle sizes in the order of micrometers to several nanometers. The electroconductive polymer is particularly preferably a conjugated polymer, and specific examples include polyaniline and polythiophene.

Furthermore, the sensitivity at the time of photocuring the resin composition for laser engraving can be further enhanced by using a co-sensitizer.

Furthermore, it is preferable to add a small amount of a thermal polymerization inhibitor in order to inhibit unnecessary thermal polymerization of the polymerizable compound during the production of the composition or during storage thereof.

For the purpose of coloration of the resin composition for laser engraving, a colorant such as a dye or a pigment may also be added. Thereby, properties such as visibility of the image unit and the suitability to the image density analyzer can be enhanced.

(Flexographic Printing Plate Precursor for Laser Engraving)

A first exemplary embodiment of the flexographic printing plate precursor for laser engraving according to the invention has a relief forming layer formed from the resin composition for laser engraving of the invention.

Furthermore, a second exemplary embodiment of the flexographic printing plate precursor for laser engraving according to the invention has a crosslinked relief forming layer obtained by crosslinking a relief forming layer formed from the resin composition for laser engraving of the invention.

The “flexographic printing plate precursor for laser engraving” according to the invention refers to both or any one of a state before the crosslinkable relief forming layer formed from a resin composition for laser engraving is crosslinked, and a state in which the relief forming layer has been cured by light or heat.

The “relief forming layer” according to the invention refers to a layer in a state of being crosslinked, that is, the relief forming layer is a layer formed from the resin composition for laser engraving of the invention, and the relief forming layer may be dried according to necessity.

By laser engraving a printing plate precursor having a crosslinked relief forming layer, the “flexographic printing plate” is produced.

The “crosslinked relief forming layer” according to the invention refers to a layer obtained by crosslinking the relief forming layer. The above-described crosslinking can be carried out by means of heat and/or light. Also, the crosslinking is not particularly limited as long as it involves a reaction by which a resin composition is cured, and the crosslinking is a concept including a crosslinked structure resulting from a reaction between Component A and Component A, or a reaction between Component D and Component D; however, it is preferable that Component A reacts with Component B to form a crosslinked structure.

Furthermore, the “relief layer” according to the invention refers to a layer engraved by laser in a flexographic printing plate, that is, a laser-engraved crosslinked relief forming layer.

The resin composition for laser engraving of the invention can also be widely used in applications other than the application of forming a relief forming layer of a flexographic printing plate precursor that is subjected to laser engraving, without any particular limitations. For example, the resin composition for laser engraving can be applied not only to the relief forming layer of a printing plate precursor, in which the formation of a convex-shaped relief is performed by laser engraving, which will be described in detail below, but also to the formation of other material shapes forming surface unevenness or openings on the surface, for example, various printing plates or various molded bodies on which necessary images are formed by laser engraving, such as an intaglio printing plate, a blank plate or a stamp.

It is preferable to produce a flexographic printing plate precursor for laser engraving by using the resin composition of the invention to form a relief forming layer on an appropriate support.

The flexographic printing plate precursor for laser engraving according to the invention has a relief forming layer formed from a resin composition for laser engraving including components such as described above. It is preferable that the (crosslinked) relief forming layer is provided on a support.

The flexographic printing plate precursor for laser engraving may further have an adhesive layer between a support and a (crosslinked) relief forming layer as necessary, and may also have a slip coat layer and a protective film on the (crosslinked) relief forming layer.

<Relief Forming Layer>

The relief forming layer is a layer formed from the resin composition for laser engraving of the invention, and is preferably a thermally crosslinkable layer.

Regarding an embodiment for producing a flexographic printing plate using a flexographic printing plate precursor for laser engraving, an embodiment in which a flexographic printing plate is produced by crosslinking a relief forming layer to obtain a flexographic printing plate precursor having a crosslinked relief forming layer, and then laser-engraving the crosslinked relief forming layer (hard relief forming layer) to form a relief layer, is preferred. By crosslinking the relief forming layer, abrasion of the relief layer at the time of printing can be prevented, and a flexographic printing plate having a relief layer with a sharp shape can be obtained after laser engraving.

The relief forming layer can be formed by molding a resin composition for laser engraving having components for relief forming layer such as described above, into a sheet form or a sleeve shape. The relief forming layer is usually provided on a support that will be described below; however, the relief forming layer can also be formed directly on the surface of a member such as a cylinder included in an apparatus for plate-making or printing, or can be disposed and then fixed thereon. Thus, a support is not essentially needed.

In the following, the invention will be explained by taking a case in which the relief forming layer is mainly produced into a sheet form, as an example.

<Support>

The material used for the support of the flexographic printing plate precursor for laser engraving is not particularly limited; however, a material having high dimensional stability is preferably used. Examples thereof include metals such as steel, stainless steel, and aluminum; plastic resins such as polyesters (for example, PET (polyethylene terephthalate), PBT (polybutylene terephthalate), PAN (polyacrylonitrile)), and polyvinyl chloride; synthetic rubbers such as styrene-butadiene rubber; and plastic resins (an epoxy resin or a phenolic resin) reinforced with glass fibers. As the support, a PET film or a steel substrate is preferably used. The form of the support is determined on the basis of whether the relief forming layer is in a sheet form or a sleeve form.

<Adhesive Layer>

In a case in which the relief forming layer is formed on a support, an adhesive layer may be provided between the two layers for the purpose of strengthening the interlayer adhesive force.

Regarding the material (adhesive) that can be used for the adhesive layer, for example, those materials described in I. Skeist, ed., “Handbook of Adhesives”, 2^{nd} Edition (1977), can be used.

<Protective Film and Slip Coat Layer>

For the purpose of preventing scratches or depressions on the surface of the relief forming layer or the surface of the crosslinked relief forming layer, a protective film may be provided on the surface of the relief forming layer or on the surface of the crosslinked relief forming layer. The thickness of the protective film is preferably 25 μm to 500 μm, and more preferably 50 μm to 200 μm. For the protective film, for example, a polyester-based film such as PET, or a polyolefin-based film such as PE (polyethylene) or PP (polypropylene) can be used. Furthermore, the surface of the film may be mattified. It is preferable that the protective film is releasable.

In a case in which the protective film cannot be released, or on the contrary, in a case in which the protective film is not easily adhered to the relief forming layer, a slip coat layer may be provided between the two layers. Regarding the material used for the slip coat layer, it is preferable to use a resin that can be dissolved or dispersed in water and is less sticky, such as polyvinyl alcohol, polyvinyl acetate, a partially saponified polyvinyl alcohol, hydroxyalkyl cellulose, alkyl cellulose, or a polyamide resin, as a main component.

(Method for Producing a Flexographic Printing Plate Precursor for Laser Engraving)

The method for producing a flexographic printing plate precursor for laser engraving is not particularly limited; however, for example, a method of preparing a resin composition for laser engraving, removing the solvent from this coating liquid composition for laser engraving as necessary, and then melt extruding the coating liquid composition for laser engraving on a support, may be used. Alternately, a method of casting a resin composition for laser engraving on a support, drying this in an oven, and removing the solvent from the resin composition, may also be used.

Among them, the method for producing a flexographic printing plate precursor for laser engraving according to the invention is preferably a production method including a layer forming step of forming a relief forming layer formed from the resin composition for laser engraving of the invention, and a crosslinking step of obtaining a flexographic printing plate precursor having a crosslinked relief forming layer, which is produced by crosslinking the relief forming layer by means of heat and/or light.

Thereafter, if necessary, a protective film may be laminated on the relief forming layer. The lamination can be performed by pressing the protective film and the relief forming layer using a heated calender roll or the like, or by adhering the protective film to the relief forming layer which has been impregnated with a small amount of a solvent on the surface.

In the case of using a protective film, a method of first laminating the relief forming layer on the protective film, and then laminating a support thereon may be employed.

In a case in which an adhesive layer is provided, this can be achieved by using a support having an adhesive layer applied thereon. In a case in which a slip coat layer is provided, this can be achieved by using a protective film having a slip coat layer applied thereon.

<Layer Forming Step>

It is preferable that the method for producing a flexographic printing plate precursor for laser engraving according to the invention includes a layer forming step of forming a relief forming layer formed from the resin composition for laser engraving of the invention.

Preferred examples of the method for forming a relief forming layer include a method of preparing the resin composition for laser engraving of the invention, removing the solvent from this resin composition for laser engraving as necessary, and then melt extruding the resin composition for laser engraving on a support; and a method of preparing the resin composition for laser engraving of the invention, casting the resin composition for laser engraving of the invention on a support, drying this in an oven, and thereby removing the solvent.

The resin composition for laser engraving can be produced by, for example, dissolving or dispersing Component A and Component B, and Component C to Component G as optional components in an appropriate solvent, and subsequently mixing these liquids. Since it is preferable to remove most of the solvent component in the stage of producing the flexographic printing plate precursor, it is preferable to suppress the total amount of addition of the solvent to the minimum level by using a low molecular weight alcohol that is highly volatile (for example, methanol, ethanol, n-propanol, isopropanol, or propylene glycol monomethyl ether) or the like as the solvent, and adjusting the temperature or the like.

Furthermore, it is also preferable to prepare the resin composition for laser engraving by kneading Component A and Component B, and Component C to Component G as optional components, using a Plastomill or the like, without using a solvent.

The thickness of the (crosslinked) relief forming layer in the flexographic printing plate precursor for laser engraving is preferably from 0.05 mm to 10 mm, more preferably from 0.05 mm to 7 mm, and even more preferably from 0.05 mm to 3 mm, before and after crosslinking.

<Crosslinking Step>

The method for producing a flexographic printing plate precursor for laser engraving according to the invention is preferably a production method including a crosslinking step of obtaining a flexographic printing plate precursor having a crosslinked relief forming layer that has been obtained by crosslinking the relief forming layer by means of heat and/or light, and the method is more preferably a production method including a crosslinking step of obtaining a flexographic printing plate precursor having a crosslinked relief forming layer that has been obtained by crosslinking the relief forming layer by means of heat.

In the present step, there is a risk that inhibition of polymerization may occur in the presence of oxygen. Therefore, crosslinking may be performed in a state in which at least the central section of the surface of the relief forming layer is covered with a material which is capable of blocking air. Also, it is preferable to perform crosslinking in a state in which the relief forming layer is tightly sealed by a material which is capable of blocking air.

In addition to that, crosslinking may be performed in a state in which the surface of the relief forming layer is brought into direct contact with air, or crosslinking may also be performed in a state in which the relief forming layer is wrapped with a sheet of a material capable of blocking air, such as vinyl chloride, and a vacuum is drawn.

The relief forming layer can be crosslinked by heating the flexographic printing plate precursor for laser engraving (thermal crosslinking step). Examples of the heating means for performing heat-induced crosslinking include a method of heating the printing plate precursor in a hot air oven or a far-infrared oven for a predetermined time; and a method of bringing the printing plate precursor into contact with a heated roll for a predetermined time.

When the relief forming layer is thermally crosslinked, there are advantages that firstly, the relief formed after laser engraving becomes sharp, and secondly, the stickiness of the laser engraving generated at the time of laser engraving is suppressed.

Furthermore, since a crosslinked structure is formed by polymerizing polymerizable compounds using a photopolymerization initiator or the like, it is also acceptable to further perform light-induced crosslinking.

In a case in which the relief forming layer contains a photopolymerization initiator, the relief forming layer can be crosslinked by irradiating the relief forming layer with the light that triggers the photopolymerization initiator (also referred to as “active light ray”).

It is common to perform irradiation with light over the entire surface of the relief forming layer. Examples of the light include visible light, ultraviolet radiation, and an electron beam; however, ultraviolet radiation is most commonly used. If the side of the substrate for fixing the relief forming layer, such as the support of the relief forming layer, is designated as a back surface, it is sufficient to irradiate the front surface with light; however, if the support is a transparent film capable of transmitting active light rays, it is preferable to irradiate the relief forming layer with light even through the back surface. In a case in which a protective film is present, irradiation through the front surface may be performed in a state of having the protective film provided thereon, or irradiation may be performed after the protective film is detached. Since there is a risk that inhibition of polymerization may occur in the presence of oxygen, irradiation with active light rays may be performed after wrapping the relief forming layer with a vinyl chloride sheet and drawing a vacuum.

(Flexographic Printing Plate and Plate-Making Method Thereof)

It is preferable that the method for making a flexographic printing plate according to the invention includes a step of preparing the flexographic printing plate precursor for laser engraving according to the invention; and an engraving step of laser-engraving this flexographic printing plate precursor for laser engraving.

The flexographic printing plate according to the invention is obtained by plate-making according to the method for making a flexographic printing plate as described above.

<Engraving Step>

It is preferable that the method for making a flexographic printing plate according to the invention includes an engraving step of laser-engraving the flexographic printing plate precursor having a crosslinked relief forming layer.

The engraving step is a step of laser-engraving the crosslinked relief forming layer that has been crosslinked in the crosslinking step, and thereby forming a relief layer. Specifically, it is preferable to form a relief layer by performing engraving by irradiating the crosslinked relief forming layer that has been crosslinked, with laser light corresponding to a desired image. Also, a process of controlling the laser head with a computer based on the digital data of a desired image, and scan-irradiating the crosslinked relief forming layer, may be preferably employed.

In this engraving step, an infrared laser is preferably used. When the crosslinked relief forming layer is irradiated with infrared laser light, the molecules in the crosslinked relief forming layer undergo molecular vibration, and heat is generated. When a high power laser such as a carbon dioxide laser or a YAG laser is used as the infrared laser, a large amount of heat is generated in the part irradiated with laser light, the molecules in the crosslinked relief forming layer undergo molecular cleavage or ionization, and thus selective removal, that is, engraving, is achieved. An advantage of laser engraving is that since the engraving depth can be arbitrarily set, the structure can be controlled three-dimensionally. For example, in an area where fine half-tone dots are printed, the relief can be prevented from falling down due to the printing pressure, by engraving the area to a shallow depth or by attaching shoulders; and when a grooved area where fine outline characters are printed is engraved deeply, grooves are not easily filled with ink, and thus the outline characters are prevented from collapsing.

Above all, in a case in which engraving is performed using an infrared laser light having a wavelength equivalent to the absorption wavelength of the photothermal conversion agent, selective removal of the crosslinked relief forming layer can be achieved with higher sensitivity, and thus a relief layer having a sharp image can be obtained.

The infrared laser used for the engraving step is preferably a carbon dioxide laser (CO₂ laser) or a semiconductor laser, in view of productivity, cost, and the like. Particularly, fiber-coupled semiconductor infrared laser (FC-LD) is preferably used. Generally, a semiconductor laser enables laser oscillation with high efficiency compared to a CO₂ laser, and size reduction can be achieved at low costs. Furthermore, since semiconductor lasers are small-sized, arraying of lasers can be easily achieved. Also, the beam shape can be controlled by a treatment of fibers.

Regarding the semiconductor laser, laser light having a wavelength of 700 nm to 1,300 nm is preferred; laser light having a wavelength of 800 nm to 1,200 nm is more preferred; laser light having a wavelength of 860 nm to 1,200 nm is even more preferred, and laser light having a wavelength of 900 nm to 1,100 nm is particularly preferred.

Furthermore, a fiber-coupled semiconductor laser can efficiently output laser light by further attaching optic fibers, and therefore, a fiber-coupled semiconductor laser is effective for the engraving step according to the invention. Furthermore, the beam shape can be controlled by a treatment of fibers. For example, the beam profile can be adjusted to a top hat shape, and energy can be stably applied to the printing plate surface. The details of semiconductor lasers are described in “Laser Handbook, 2^{nd} Edition” edited by the Laser Society of Japan, “Practical Laser Technology” edited by the Institute of Electronics and Communication Engineers of Japan, and the like.

Furthermore, a plate-making apparatus equipped with a fiber-coupled semiconductor laser, which can be suitably used for the method for making a flexographic printing plate using the flexographic printing plate precursor according to the invention, is described in detail in JP2009-172658A and JP2009-214334A, and this can be used for the plate-making of the flexographic printing plate related to the invention.

<Other Steps>

The method for making a flexographic printing plate according to the invention may further include a rinsing step, a drying step and/or a post-crosslinking step described below as necessary, subsequently to the engraving step.

Rinsing step: a step of rinsing the engraved surface of the relief forming layer after engraving with an aqueous rinsing liquid.

Drying step: a step of drying the relief layer after the rinsing step.

Post-crosslinking step: a step of applying energy to the relief layer after engraving, and further crosslinking the relief layer.

After going through the above-described steps, since engraving residue is adhering to the engraved surface, a rinsing step of rinsing the engraved surface with an aqueous rinsing liquid (hereinafter, also simply referred to as “rinsing liquid”), and thereby washing off the engraving residue, may be added. The aqueous rinsing liquid is water, or a liquid containing water as a main component. Examples of the rinsing means include a method of washing with tap water; a method of spraying high pressure water; and a method of rubbing the engraved surface with a brush mainly in the presence of water, using a batch type or conveyance type brush washing machine, which is known as a developing machine for a photosensitive resin letterpress printing plate. In a case in which the slime of the engraving residue cannot be removed, a rinsing liquid containing soap or a surfactant may be used.

In a case in which the rinsing process of rinsing the engraved surface is implemented, it is preferable to add a drying step of drying the engraved relief forming layer, and thereby volatilizing the rinsing liquid.

Furthermore, a post-crosslinking step of further crosslinking the relief forming layer may also be added as necessary. By implementing a post-crosslinking process, which is an additional crosslinking process, the relief formed by engraving can be further strengthened.

The pH of the rinsing liquid that can be used for the invention is preferably 9 or higher, more preferably 10 or higher, and even more preferably 11 or higher. Furthermore, the pH of the rinsing liquid is preferably 14 or lower, more preferably 13.5 or lower, even more preferably 13.2 or lower, and particularly preferably 12.5 or lower. When the pH is in the above-mentioned range, handling thereof is made easier.

In order to adjust the rinsing liquid to the pH range described above, the pH may be adjusted by appropriately using an acid and/or a base, and the acid and base to be used are not particularly limited.

It is preferable that the rinsing liquid that can be used for the invention contains water as a main component.

Also, the rinsing liquid may include a water-miscible solvent such as an alcohol, acetone, or tetrahydrofuran, as a solvent other than water.

It is preferable that the rinsing liquid contains a surfactant.

From the viewpoint of the removability of engraving residue, and from the viewpoint of reducing the influence on the flexographic printing plate, preferred examples of the surfactant that can be used for the invention include betaine compounds (amphoteric surfactants) such as a carboxybetaine compound, a sulfobetaine compound, a phosphobetaine compound, an amine oxide compound, and a phosphine oxide compound.

Furthermore, examples of the surfactant include known anionic surfactants, cationic surfactants, and nonionic surfactants. Also, fluorine-based and silicone-based nonionic surfactants can also be used similarly.

Those surfactants may be used singly or in combination of two or more kinds thereof.

It is not particularly necessary to limit the amount of use of the surfactant; however, the amount of use is preferably 0.01% by mass to 20% by mass, and more preferably 0.05% by mass to 10% by mass, relative to the total weight of the rinsing liquid.

As such, a flexographic printing plate having a relief layer on the surface of an arbitrary substrate such as a support is obtained.

The thickness of the relief layer of the flexographic printing plate is preferably from 0.05 mm to 10 mm, more preferably from 0.05 mm to 7 mm, and particularly preferably from 0.05 mm to 3 mm, from the viewpoint of satisfying various printing suitability properties such as abrasion resistance and ink transferability.

Furthermore, it is preferable that the Shore A hardness of the relief layer of the flexographic printing plate is from 50° to 90°. When the Shore A hardness of the relief layer is 50° or higher, even if the fine half-tone dots formed by engraving is subjected to a high printing pressure of a letterpress printing machine, the fine half-tone dots do not fall down and collapse, and normal printing can be carried out. Also, when the Shore A hardness of the relief layer is 90° or lower, printing blur in a solid printed area can be prevented even in the case of flexographic printing, which is performed at a kiss-touch printing pressure.

Meanwhile, the Shore A hardness according to the present specification is a value measured using a durometer (spring type rubber hardness meter), which deforms an object of measurement by pressing a depressor (referred to as a push pin or an indenter) into the surface of the object at 25° C., measures the amount of deformation thereof (indentation depth), and digitalizes the amount of deformation.

The flexographic printing plate according to the invention enables printing even in a case in which any of an aqueous ink, an oily ink, or a UV ink is used with a letterpress printing machine, and printing with a UV ink using a flexographic printing machine is also enabled.

The inventors of the present invention found that a flexographic printing plate precursor for laser engraving having excellent rinsability of the engraving residue and excellent printing durability is obtained by using a resin composition including Component A and Component B.

Although the detailed mechanism is not clearly known, in a case in which (Component A) a resin that is a plastomer, and (Component B) a vulcanizing agent are used in a resin composition, the crosslinking density of the engraving residue after laser engraving is maintained to be relatively high. Therefore, it is speculated that because the engraving residue is not easily decomposed to low molecular weight components, does not melt, and does not have viscousness, high rinsability may be obtained.

Furthermore, it is speculated that when melting of the engraving residue or the relief layer itself is suppressed, a phenomenon in which a molten material flow into the engraved section and the engraving depth becomes shallow, is suppressed, and thus the engraving sensitivity increases.

In addition, it is considered that in a case in which Component A and Component B are used in a resin composition, since the entirety of the resin composition can be sufficiently kneaded at the time of preparation, the crosslinking density of the entire relief layer becomes relatively uniform, irrespective of the position within the relief layer. It is speculated that as a result, the hardness of the relief layer also becomes relatively uniform over the entirety of the layer, a phenomenon in which at the time of printing, stress is concentrated at parts with high hardness and the relief layer is destroyed, does not easily occur, and printing durability is enhanced.

Moreover, it is considered that if the crosslinking density of the entire relief layer is uniform at the time of production of the printing plate precursor, non-uniform crosslinked sections are not easily generated within the layer even if crosslinking is slightly broken by ozone or the like that is present in air during the storage for a long time period. Therefore, it is speculated that the hardness in the relief layer is maintained in a uniform state even after long-term storage, and printing durability over time is increased.

### EXAMPLES

The present invention will be described in more detail by way of Examples; however, the present invention is not intended to be limited to these Examples. Unless particularly stated otherwise, the units “parts” and “percent (%)” represent “parts by mass” and “percent (%) by mass”, respectively. Meanwhile, the number average molecular weight (Mn) and the weight average molecular weight (Mw) of a compound in the Examples indicate values measured by a gel permeation chromatography (GPC) method (eluent: tetrahydrofuran), unless particularly stated otherwise.

The details of Component A to Component G used in the various Examples and Comparative Examples are as follows.

(Component A) Resin that is Plastomer

A-1: UBEPOL BR 150 L (manufactured Ube Industries, Ltd.), polybutadiene, Mw=467,000, Mn=243.000, Tg=about −103° C., tensile permanent strain=63%

A-2: LBR-305 (manufactured by Kuraray Co., Ltd.), Mw=28,100. Mn=26.000, polybutadiene

A-3: LIR-30 (manufactured by Kuraray Co. Ltd.), Mn=28,000, polyisoprene

A-4: TR2000 (manufactured by JSR Corporation.), styrene-butadiene rubber

(Component B) Vulcanizing Agent

B-1: Sulfur (manufactured by Tsurumi Chemical Industry Co., Ltd.)

(Component C) Compound Having Hydrolyzable Silyl Group and/or Silanol Group

C-1: Compound C-1 described below

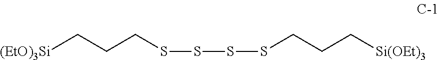

(Component D) Polymerizable Monomer

D-1: 1,6-Hexanediol diacrylate (manufactured by Sun Chemical Company LTD.)

D-2: Trimethylolpropane triacrylate (manufactured by Shin Nakamura Chemical Co., Ltd.)

(Component E) Polymerization Initiator

E-1: PERBUTYL E (manufactured by NOF Corporation., t-butylperoxy-2-ethylhexyl carbonate)

(Component F) Vulcanization Accelerating Agent

F-1: NOCCELER DM (manufactured by Ouchi Shinko Chemical Industrial Co., Ltd., di-2-benzothiazolyl disulfide)

(Component G) Photothermal Conversion Agent

G-1: KETJEN BLACK EC600JD (manufactured by Lion Corporation., carbon black)

### Examples 1 to 18 and Comparative Examples 1 to 7

### 1. Preparation of Resin Composition for Laser Engraving

In each of the Examples and Comparative Examples, an unvulcanized resin composition was obtained by kneading and blending the components at the blending composition described below, using a Labo-Plastomill.
Component A: 100 parts by mass
Component B: 12 parts by mass
Component C: indicated in Table 1
Component D: 15 parts by mass
Component E: 1.3 parts by mass
Component F: 4 parts by mass
Component G: 12 parts by mass

(Other Additives)
Stearic acid (manufactured by Wako Pure Chemical Industries, Ltd.): 1 part by mass
Zinc oxide (zinc white, manufactured by Wako Pure Chemical Industries, Ltd.): 5 parts by mass
Naphthene-based oil (manufactured by Sankyo Yuka Kogyo K.K., SNH-3): 10 parts by mass
Vanillin (manufactured by Wako Pure Chemical Industries. Ltd.): 1 part by mass

Meanwhile, the amount of use of Component C (parts by mass relative to 100 parts of Component A) was regulated as described in Table 1, and any component described as “None” in Table 1 was not added to the above-described composition.

Through this operation, coating liquids for a crosslinked relief forming layer (resin composition for laser engraving) having fluidity were respectively obtained.

### 2. Production of Flexographic Printing Plate Precursor for Laser Engraving

In a case in which A-1 to A-3 were used as Component A, a spacer (frame) having a predetermined thickness was installed on a polyethylene terephthalate (PET) substrate, the above-described resin composition thus obtained was gently cast thereon to the extent that the resin composition did not flow out over the spacer (frame), and the resin composition was subjected to thermal crosslinking for 30 minutes at 160° C. Thus, a flexographic printing plate precursor for laser engraving formed from a crosslinked relief forming layer having a thickness of approximately 1 mm was produced.

In a case in which A-4 was used as Component A, a flexographic printing plate precursor for laser engraving formed from a crosslinked relief forming layer having a thickness of approximately 1 mm was produced by performing a thermal crosslinking treatment for 30 minutes at 160° C. using a heating plate press machine.

### 3. Production of Flexographic Printing Plate by Laser Engraving

In a crosslinked relief forming layer, a solid area which measured 1 cm on each of four sides was raster-engraved by means of the following two kinds of lasers, and thus a flexographic printing plate was obtained.

As a carbon dioxide laser (CO₂ laser) engraving machine, a high-resolution CO₂ laser marker, ML-9100 Series (manufactured by Keyence Corporation.), was used to perform engraving by irradiation with a laser. After the protective film was detached from a printing plate precursor for laser engraving 1, engraving was performed with the carbon dioxide laser engraving machine under the conditions of output power: 12 W, head speed: 200 mm/sec, pitch: 2,400 DPI.

As a semiconductor laser engraving machine, a laser recording apparatus equipped with a fiber-coupled semiconductor laser (FC-LD), SDL-6390 (manufactured by JDS Uniphase Corporation., wavelength: 915 nm) having a maximum output power of 8.0 W was used. Engraving was performed using the semiconductor laser engraving machine under the conditions of laser output power: 7.5 W, head speed: 409 mm/sec, and pitch: 2,400 DPI.

The thickness of the relief layer of each of the flexographic printing plates of Examples 1 to 18 and Comparative Examples 1 to 7 thus obtained was approximately 1 mm.

### 4. Evaluation of Flexographic Printing Plates

A performance evaluation of the flexographic printing plates was carried out for the following items, and the results are presented in Table 1.

(4-1) Engraving Depth

The “engraving depth” of the relief layer obtained by laser-engraving the crosslinked relief forming layer carried by the flexographic printing plate precursor of each of the Examples and Comparative Examples was measured as follows. Here, the “engraving depth” refers to the difference between an engraved position (height) and a non-engraved position (height) in a case in which a cross-section of the relief layer was observed. The “engraving depth” according to the present Examples was measured by observing a cross-section of the relief layer with an ultra-deep color 3D profile measuring microscope VK9510 (manufactured by Keyence Corporation.). A larger engraving depth means higher engraving sensitivity. The results are presented in Table 1 separately for each kind of the laser used for engraving (carbon dioxide laser (CO₂) and fiber-coupled semiconductor laser (FC-LD)).

Meanwhile, the following evaluation of rinsability, printing durability, printing durability over time, and rupture strength was carried out using the flexographic printing plates engraved using a carbon dioxide laser.

(4-2) Rinsability

A rinsing liquid was prepared by mixing water, a 10% by weight aqueous solution of sodium hydroxide, and the betaine compound (1-B) described below, such that the pH was adjusted to 12, and the content of the betaine compound (Formula 1-B described below) was adjusted to be 1% by mass of the total amount of the rinsing liquid.

The rinsing liquid thus produced was dropped (about 100 mL/m²) with a spuit on each of the printing plate materials engraved by the above-described method such that the plate surface would be uniformly wetted. After allowing the printing plate material to stand for 30 seconds, the plate surface was rubbed 15 times (20 seconds) in the direction parallel to the plate, using a toothbrush (Clinica Toothbrush Flat normal of Lion Corporation.) under a load of 200 gf (1.96 N). Thereafter, the plate surface was washed with flowing water, moisture on the plate surface was removed, and the plate was naturally dried for about 1 hour.

Thereafter, the surface of the rinsed plate was observed with a microscope (manufactured by Keyence Corporation.) at a magnification ratio of 100 times, and the residue remaining on the plate was evaluated. The evaluation criteria were as follows.

1: No residue remained on the plate, or only a slight amount of residue remained at the bottom (concave parts) of the image.

2: A slight amount of residue remained at the convex parts of the plate image, and a slight amount of residue remained at the bottom (concave parts) of the image.

3: A slight amount of residue remained at the convex parts of the plate image, and residue remained at the bottom (concave parts) of the image.

4: Residue adhered over the entire surface of the plate.

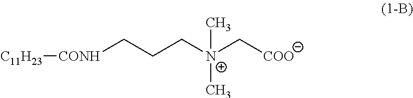

(4-3) Printing Durability

A flexographic printing plate thus obtained was mounted on a printing machine (ITM-4 type, manufactured by Iyo Kikai Seisakusho Co., Ltd.), and printing was continued using UV INK FLEXO 500CF (manufactured by T&K Toka Co., Ltd.) as an ink without diluting the ink, and using FULL COLOR FORM M70 (manufactured by Nippon Paper Industries Co., Ltd., thickness: 100 μm) as printing paper. Thus, the portion of 1% to 10% highlight was checked from the printed matters. When unprinted half-tone dots were generated, the time point was designated as completion of printing, and the length (meters) of the paper printed until the completion of printing was used as an index. A flexographic printing plate having a larger value of the index was evaluated to have superior printing durability.

(4-4) Printing Durability Over Time

A flexographic printing plate thus obtained was stored for 3 days in an atmosphere at 60° C. and a humidity of 35%, and then printing durability over time was evaluated by performing the measurement in the same manner as in the case of performing an evaluation of the printing durability. As the decrease in printing durability was smaller compared with the printing durability before storage, the planographic printing plate precursor was evaluated to have superior printing durability over time.

(4-5) Rupture Strength

For a cured film (relief layer) obtainable by curing the resin composition for laser engraving of each of the Examples and Comparative Examples, the rupture strength was measured as follows.

The rupture strength was measured by processing a specimen to have the dumbbell shape described in the JIS Standards (measurement was made by inputting the average of horizontal width to be 2.25 cm), using Shimadzu AGSH5000 manufactured by Shimadzu Corporation, as a tensile testing machine. The measurement environment was set to the following: temperature: about 21° C., humidity: 60%, and tensile rate: 2 mm/min. A relief layer having a larger value was evaluated to have superior strength.

TABLE 1

| | | | | | | | | | | | | | Priming | |
|---|---|---|---|---|---|---|---|---|---|---|---|---|---|---|
| | | | | | | | | | CO₂ | FC-LD | | Printing | dura- | |
| | | | | Parts by | | | | | engrav- | engrav- | | dura- | bility | |
| | | | | mass of | | | | | ing | ing | Rins- | bility | over time | Rupture |
| | Compo- | Compo- | Compo- | Compo- | Compo- | Compo- | Compo- | Compo- | depth | depth | abil- | (UV ink) | (UV ink) | strength |
| Example | nent A | nent B | nent C | nent C | nent D | nent E | nent F | nent G | (μm) | (μm) | ity | (m) | (m) | (N/cm²) |
| Example 1 | A-1 | B-1 | C-1 | 0.05 | D-1 | E-1 | None | None | 300 | 0 | 2 | 18,000 | 18,000 | 16 |
| Example 2 | A-1 | B-1 | C-1 | 0.05 | D-1 | E-1 | F-1 | None | 310 | 0 | 1 | 19,000 | 19,000 | 17 |
| Example 3 | A-1 | B-1 | C-1 | 0.05 | D-1 | E-1 | None | G-1 | 320 | 400 | 1 | 20,000 | 20,000 | 19 |
| Example 4 | A-1 | B-1 | C-1 | 0.05 | D-1 | E-1 | F-1 | G-1 | 330 | 410 | 1 | 22,000 | 22,000 | 22 |
| Example 5 | A-1 | B-1 | C-1 | 0.01 | D-1 | E-1 | F-1 | G-1 | 330 | 410 | 1 | 22,000 | 22,000 | 21 |
| Example 6 | A-2 | B-1 | C-1 | 0.05 | D-1 | E-1 | F-1 | G-1 | 335 | 410 | 1 | 20,000 | 20,000 | 17 |
| Example 7 | A-2 | B-1 | C-1 | 0.05 | D-1 | E-1 | F-1 | G-1 | 340 | 415 | 1 | 21,000 | 21,000 | 18 |
| Example 8 | A-1/A-2 | B-1 | C-1 | 0.04 | D-1 | E-1 | None | None | 305 | 0 | 2 | 17,000 | 17,000 | 14 |
| Example 9 | A-1/A-2 | B-1 | C-1 | 0.04 | D-1 | E-1 | F-1 | None | 310 | 0 | 2 | 18,000 | 18,000 | 15 |
| Example 10 | A-1/A-2 | B-1 | C-1 | 0.04 | D-1 | E-1 | None | G-1 | 322 | 405 | 1 | 21,000 | 21,000 | 16 |
| Example 11 | A-1/A-2 | B-1 | C-1 | 0.04 | D-1 | E-1 | F-1 | G-1 | 331 | 410 | 1 | 22,000 | 22,000 | 18 |
| Example 12 | A-3 | B-1 | C-1 | 0.06 | D-1 | E-1 | None | None | 295 | 0 | 2 | 16,000 | 16,000 | 12 |
| Example 13 | A-3 | B-1 | C-1 | 0.06 | D-1 | E-1 | F-1 | None | 300 | 0 | 2 | 17,000 | 17,000 | 13 |
| Example 14 | A-3 | B-1 | C-1 | 0.06 | D-1 | E-1 | None | G-1 | 298 | 390 | 1 | 18,000 | 18,000 | 13 |
| Example 15 | A-3 | B-1 | C-1 | 0.06 | D-1 | E-1 | F-1 | G-1 | 303 | 395 | 1 | 19,000 | 19,000 | 15 |
| Example 16 | A-1 | B-1 | C-1 | 0.05 | D-2 | E-1 | F-1 | C-1 | 320 | 400 | 1 | 21,000 | 21,000 | 23 |
| Example 17 | A-1 | B-1 | C-1 | 0.05 | None | E-1 | F-1 | G-1 | 320 | 400 | 1 | 22,000 | 22,000 | 20 |
| Example 18 | A-1 | B-1 | C-1 | 0 | D-1 | E-1 | F-1 | G-1 | 330 | 410 | 2 | 22,000 | 22,000 | 20 |
| Comparative | A-1 | None | C-1 | 0.05 | D-1 | E-1 | None | None | 280 | 0 | 4 | 13,000 | 13,000 | 14 |
| Example 1 | | | | | | | | | | | | | | |
| Comparative | A-1 | None | C-1 | 0.05 | D-1 | E-1 | None | G-1 | 285 | 340 | 3 | 15,000 | 15,000 | 15 |
| Example 2 | | | | | | | | | | | | | | |
| Comparative | A-1 | None | C-1 | 0.2 | D-1 | E-1 | None | G-1 | 285 | 340 | 3 | 15,000 | 7,000 | 15 |
| Example 3 | | | | | | | | | | | | | | |
| Comparative | A-1/A-2 | None | C-1 | 0.04 | D-1 | E-1 | None | G-1 | 287 | 350 | 3 | 16,000 | 16,000 | 11 |
| Example 4 | | | | | | | | | | | | | | |
| Comparative | A-3 | None | C-1 | 0.06 | D-1 | E-1 | None | G-1 | 290 | 335 | 3 | 14,000 | 14,000 | 12 |
| Example 5 | | | | | | | | | | | | | | |
| Comparative | A-4 | B-1 | C-1 | 0 | D-1 | E-1 | F-1 | G-1 | 320 | 370 | 4 | 22,000 | 22,000 | 30 |
| Example 6 | | | | | | | | | | | | | | |
| Comparative | A-1 | None | C-1 | 0.2 | D-1 | E-1 | F-1 | G-1 | 290 | 350 | 3 | 16,000 | 10,000 | 17 |
| Example 7 | | | | | | | | | | | | | | |

What is claimed is:

1. An artificial nail removal method comprising:
a step of applying an artificial nail composition on or above the nail of a human being or an animal, or on or above a support to form a coating film;
a step of exposing to light the coating film, and thereby forming an artificial nail; and
a removal step of removing the artificial nail by bringing the artificial nail into contact with a removal liquid,
wherein the artificial nail composition includes:
(Component A) a compound having an ethylenically unsaturated group and an acid group, and/or (Component C) a polymerizable compound that does not have an acid group;
(Component B) a polymer having an acid group; and
(Component D) a photopolymerization initiator,
Component B is an acrylic polymer containing a monomer unit represented by the following Formula (2) or a urethane polymer having a carboxylic acid group, and
the removal liquid is an aqueous solution having a pH of from 8 to 11, 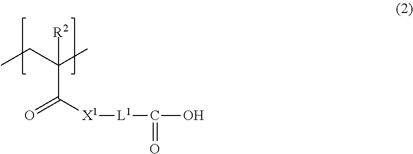
wherein in Formula (2), R² represents a hydrogen atom, a methyl group, or —CH₂Y¹; Y¹ represents a hydroxyl group, a halogeno group, —OC(═O)R¹, or —NR³C(═O)R⁴; X¹ represents —O— or —NR³—; R³ and R⁴ each represent a hydrogen atom, an alkyl group having 1 to 6 carbon atoms, or an aryl group having 6 to 12 carbon atoms; and L¹ represents a divalent linking group.

2. The artificial nail removal method according to claim 1, wherein the artificial nail composition includes Component A.

3. The artificial nail removal method according to claim 2, wherein Component A is represented by the following Formula (1): 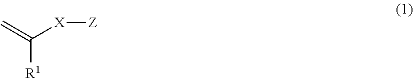
wherein in Formula (1), R¹ represents a hydrogen atom or a monovalent organic group; X represents a single bond or a divalent linking group; and Z represents an acid group.

4. The artificial nail removal method according to claim 3, wherein in the above Formula (1), Z represents a carboxylic acid group.

5. The artificial nail removal method according to claim 1, wherein Component B is an acrylic polymer containing a monomer unit represented by the above Formula (2).

6. The artificial nail removal method according to claim 1, wherein Component B is a urethane polymer having a monomer unit represented by the following Formula (3): 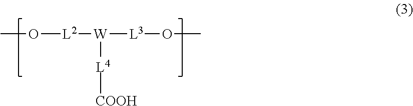
wherein in Formula (3), L², L³ and L⁴ each independently represent a divalent linking group; W represents N or CR³; and R³ represents a hydrogen atom or a monovalent group.

7. The artificial nail removal method according to claim 1, wherein the removal liquid includes a pH buffering agent.

8. The artificial nail removal method according to claim 7, wherein the pH buffering agent includes at least one selected from the group consisting of a carbonate, a hydrogen carbonate, a phosphate, a borate, and an organic amine compound.

9. The artificial nail removal method according to claim 7, wherein the pH buffering agent includes a carbonate and a hydrogen carbonate.

10. The artificial nail removal method according to claim 1, wherein the removal step is a step of removing the artificial nail by immersing the artificial nail in a removal liquid.

11. The artificial nail removal method according to claim 1, wherein the removal step is a step of removing the artificial nail by spraying a removal liquid to the artificial nail.

12. The artificial nail removal method according to claim 1, wherein the removal step is a step of removing the artificial nail by wrapping the artificial nail using cotton soaked with a removal liquid.

13. The artificial nail removal method according to claim 1, wherein the artificial nail composition is used as a base layer.

14. An artificial nail composition comprising:
(Component A) a compound having an ethylenically unsaturated group and an acid group, and/or (Component C) a polymerizable compound that does not have an acid group;
(Component B) a polymer having an acid group; and
(Component D) a photopolymerization initiator,
Component B is an acrylic polymer containing a monomer unit represented by the following Formula (2) or a urethane polymer having a carboxylic acid group, 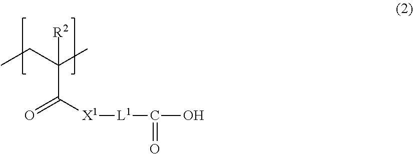
wherein in Formula (2), R² represents a hydrogen atom, a methyl group, or —CH₂Y¹; Y¹ represents a hydroxyl group, a halogeno group, —OC(═O)R³, or —NR³C(═O)R⁴; X¹ represents —O— or —NR³—; R³ and R⁴ each represent a hydrogen atom, an alkyl group having 1 to 6 carbon atoms, or an aryl group having 6 to 12 carbon atoms; and L¹ represents a divalent linking group.

15. The artificial nail composition according to claim 14, wherein Component B is an acrylic polymer containing a monomer unit represented by the above Formula (2).

16. The artificial nail composition according to claim 14, wherein Component B is a urethane polymer having a monomer unit represented by the following Formula (3): 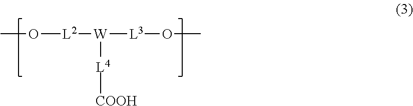
wherein in Formula (3), L², L³, and L⁴ each independently represent a divalent linking group; W represents N or CR³; and R³ represents a hydrogen atom or a monovalent group.

17. An artificial nail forming method comprising:
a step of applying the artificial nail composition according to claim 14 on or above the nail of a human being or an animal, or on or above a support to form a coating film; and
a step of exposing to light the coating film, and thereby forming an artificial nail.

18. A nail art kit comprising:
an artificial nail composition; and
a removal liquid,
wherein the artificial nail composition includes:
(Component A) a compound having an ethylenically unsaturated group and an acid group, and/or (Component C) a polymerizable compound that does not have an acid group;
(Component B) a polymer having an acid group; and
(Component D) a photopolymerization initiator,
Component B is an acrylic polymer containing a monomer unit represented by the following Formula (2) or a urethane polymer having a carboxylic acid group, and
the removal liquid is an aqueous solution having a pH of from 8 to 11, 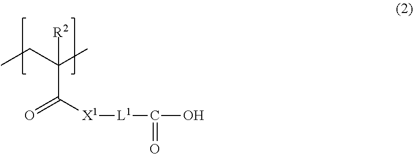
wherein in Formula (2), R² represents a hydrogen atom, a methyl group, or —CH₂Y¹; Y¹ represents a hydroxyl group, a halogeno group, —OC(═O)R³, or —NR³C(═O)R⁴; X¹ represents —O— or —NR³—; R³ and R⁴ each represent a hydrogen atom, an alkyl group having 1 to 6 carbon atoms, or an aryl group having 6 to 12 carbon atoms; and L¹ represents a divalent linking group.
